SEQUENCE LISTING (31 sequences)

SEQ ID NO: 1 - DNA, 9075 bp, Artificial Sequence
  Description of Artificial Sequence Synthetic polynucleotide
ggcaagttgatatactggctctgatcacggactattctggtgttggtggacactctctccacaagtaggcgccgtggagttcatgctcgttctgaaccgggaatggcacaacccgtagtaggatcttgcctctgccacgtgcacctgcgcctgttgcttctagactatgggctgctaaggatgatcacagactcctttttggacaatctgtgtcaagtctactgttacatctgatgtacgctggaaatcctcgtactggaggcaatacctggaggtggtgctgaaatattgcaagccacaggtgtgtttctagtcttggtgccgctatcagatgtcgcaccctctacgggcaaatgtagaaccttagtggacgcgtgagatgggaatccacgcctggccatagactggctgataagctcgcggctgatcgagttgcaacagtaatcagttgatttgccactatgtcgtacaaacctgaagataatgctccactccagtctcaacaagaggataaagcaccctctgtagctccacatgagaaagccactgtggagacaacagttgctgctgttgtacagagtgaacaggatgtcatttttgataattcatggacggatgttaggacaattgatgcagaaattcctaggatagttgctacctactcatggacttctactcaagcatccggtgacttcctcacagctgttacaggttccgatgctgttgaaaccctcccttcctccccgaaccctttctgccttccgaattacttaattgtcaatcagcccgcttcttctagtggtcttaatgtccctgcacaggtaggcctattccaagccctcttcacgaggcatgccctctataggtcaggctttagggttgaagttactgttaacggaacccagtttcatggtggttctctctttgtagttgcaattccacatccccacgtctggatgcaatcacttacaaatacagacgcgggcactcaaaagaagtggactgcccttgagcttgtacaggtcaatcagcttggcatctttccttcagcaagacttctccccagaaatgcttccgctgttgctcttgatctcccctatgttgggcctgcgcctcaattggagactgctgctcttgatgtgatgtatgcaattgctattcttgtagagactcctctctccatacctacaggaactgcccccactctcactgtggtaatgcgagtcggtgctactaatgcacatttctttgcccccaaaccctaccagactccccaatctaatcttggtgcttttgatgtgcctttggatgtccccgccccagcctcgttgctgcaaggggtgcgtgtgaacactgcgcaatctgcgtttgttaacacgtcttctggatcgatagcacagattgagagacggtcaagaagtaccgaaacaacttttcttcctgcacgagtagaggactggaggtctattctttccaggcccactttgatgtctcttgtccaattcacagctgccacaacccttgggacgaacatctttcagacaaatgtgtgtccaactggaaccagtgttacgaatcgtgacgccacgcaagcactcccaaatgcttccatcaagcgtcttcaaggtacaccttatctctcacagcttgcgcgctacttctcccagtggcgcgggtctctcacatatacatttgagtacactggccctcaggtctcttctgggagacttcttattgccttcttgccggggagagtccgggcagcgaaatctggacaaggagcagaatatgtggagactggagcacttttgcagaactttacagaaaaccttcatgtgatctgggatctttcaaattctacctctgtctcaatcacttgtccttatgctattgctaccccctgggctccaatcgtgatcaactcatggcaggcaattcccctttcaggctattcctcgggaactcttgtggctgttgtgatgtcacctcttgttactccaactgttgttgccccgacatccacaattttagtgcatctttctgctggttcagattttgaaatgcgcttccccgccccaattcctgtaaataccattcccatgtataatggtattgtagatcaaggcccagctgtggacgatctcccccaaggtgagcattatctcacagctatggacacaatgtctcttcagactttcttcaaccagtcaagattctatgggaatttttctgccactgccggtacaactgcttcaatggtcacaatccccctgtcactcatttcattcgcaccctcttctaatcttaaagtaccagaagcccccgtgcgaatgtttgcaggtctctttacttttcttcgcgctgacctaagaataactgtgggaatacccttccagcacaacttgattgttacataccgtccacctactgccaccccttccacctacagtgcagctggtgcaggagctcccgattctgccgcctcgcttctgaatggtccctctgtgattctctcaacccgcgcttcaaatatgggtgtggagtttgtgataccattctctgcttttgcgtctgtttttcagacctcctggtcacaggccaaccgggtggataccaggtataatccagactccaaccagaccaatcctggtgatatgggtactatctttgttgcctcccgcctccccagtgctcagctttctaatgtctctgtttttattgcgtttcagaatgcagaattctttgttccccgtcccttcccccccctcacttcgactgggtgggatggcacagcccccaataatttggagggggagttttgggaggaggaggacccaactccagtggagttccaagccccaaccactgaaggtctagaaaagggtgatcaggtcatgttgtctgttactttggacattctggattcaatcagggagctttgttcagaactcatggaaaatgtagacactttgtatggccgagcgcgggcagcaaagttcgcttggttggttcagagtacttttgaagaatggaagaagaatgaccaagatgctgatgagttcttctctgatgtctctcttgtggagccagttgtcttccaatcacccccttccccctctgctagagaaaaagctgctcggcttgaagaattctttgattacatgtatggatgtgtggaaagtgaggaggaattgcatgctgtgtgtgatgctgtgaatgctgtgattgactctttttatgattctgatggtgactttattggtgaaattagagatagattccttgaactccccctttcctatcctgaaggtcctgatcacgctggtgtttggtttagaggggaattctaccgttgcctctatgttacaactcaaaatcggttcttcccacaggttgctgcctattctcctgttgaaactgaacacaacctttttgcgctctatgaggcactgagacagtttgaactgatgtgtattgctgatcccctctctacttcccaccattatcagaggctccttttcctgcttctgcaacagcaatgggtcattcctgaaatgaagtcaatggttagagaaaaactagccttcctctacaccatggttgcacaccctgaacgtatttttcttgactggaattgggaacttttccacacccagccattccgttacatttgcaatatggttggagtggaaactgccttgaccgatgatggctttttctcgggtgatgaagaaggccaattccaagcaccaacatcctgtgatcagaaatttttgaacttggtcagggcctacagagatgcaatgattgtaaacccctggcacacctttgaagtctgttacatttctttttatgatctaacctgtaggagtgatgtgccagaatggtttaaaagagctgttgaggatgcttgctttgataccctgtgtgcgagagttgctgagcgagtgaatgttaataatgaagtcttgattgagagatatagacagctgtatgatgaaatcttttctgaggaacctgatttcttttctgatgatgagattgatgctgaattccaagcgcccaaaccccccgctccagcttcctcggttcctgtcccgcgcccctcaacacccctcccctctcctgctacttcagaagatgaagatgaaggtgaagatgagcccgatgatatatgtgtctactatgtagacaatgggctttaccgccattggggaattcgcaataaacaacaggcgatttctctcaaggcacatggctttcagtgtcgtgtttcatatacacctgaagatcttggaaagcatgtggactattgtactgttgggtacaatgagtggtgtaaggctgtttgcagattgggtcaaatttatgattattcaattacgcacaattgcactgattttgtccaagaaattacttcttaccagcattccttccaaaacactggctattggcttgctgctggtgtggttgctgctgctgttgctacaactgctatttttcattctgtccctccttcctgggatgagaggccatttctacccgagaaaacgaccccgagggtagtagtggtacctccccgttgtagggttcacagatggagaagggcaacgcttgagaaaattatagaactcaagagaaagatgcttttccaggcaccaataaacatccaacttactaatcctgctgttgatagagctgcctcagttacaacatctgcgactcttgaaacacttgatgaggtcagaaatatggtgcccgttttagttaattctgcttctactgtagcttcttctctctctgctgcctctgcttccgttgagcatctcacaaaatcaatggagacctttcttgaaaatgctacttctgctattcctggtgctgtctctgcaatggaatccactgctggagagctcatttcttctgcttctaagaaggttgcctcaattattctcaaagtgattggttttactctagtaatttttggaaatccctccccctcgacaatagcgggtgtgatcgctcttttggttgctgaggtctatgataatgcttatcttaggaaaaagatgtctgatctggctgtctcctttacttctaaaattaaggatcttgtcggttctctctttggtgtttctagctgctgtgatgaagaacccatctttactgatattgatgatgctttccatcgctacatgcttacccgcactgagactgacttcgaattccaagccccatctcgccttcaagagtttaatcagggtgtccttgcaatgaagaatgttgaatggataattgaaaagatcaaagaactcattgattggctcatttccaaactcagatctaaggaaaggtcctcacctgaagactatctcaaaactagggctgattacatcactgatctctatcgtgattcccttgccacagcctcttgccagaatgttgatgtgaatcttttgaatcagcgcgttagagacactagagatatgcttgcttactgcacaaatcacagacttgctggtgctgctcatatcctctctcgcactcttgctaattacgagcaaacttctagaaagcttaaaacttctacctattcctcccgccctgaaccccttgtgatttatatccatggttctcctggtgttggtaaatctcttctctctaatataattgcttcagcttattgcaagagaaagggaattgatttcaagacctccgtctttgcccaacctcctggttctgaatactttgatggttacactgggcagcctgtccacgtgattgatgacttttgccagaacactaccggtgaggatgtgaaacttttctgtcagatggtctccactactccttttgttcccccaatggctgctcttgaagagaaaggcgtccaatatcgatcccgcctcattcttgctacctccaatatggcaactccccaatcgaacgagatacggattcccggtgcccttgaacgccgctgccatatcaaagtgcgtgccattttggcgcgcgctttccagacccccgctggtcggttggatatggctgaagctttcaaggaacttggacctgcaaagtcgcccgacttcaaggcagattgtccctatcttaacggagctgctatgactttgtctgttacaaaaggtgaggatcgtactcgtgagcacatgtccgtttatgatctttgtgatatgatttttgatgaacttgatgagcgtgatgggaatcagactgccttttctaaaatcattttccaagctccctcgcagaacgtaatttctttctgtaagcatgctgatcactcacccgattcctgctacaatatcttcattgctactccccaaggaccaattgtgcatagatttgattctgtagaggataggaattcctggcttcagtctcaaattccttctcttccaaaagctgggaatgttaggtgttgtccctgtaaaaatcctgattgtgcaaagatccaattcactttcaagactgatgatgagcaaattcgatcccaggttattaactttcctggacgcgatgttgcagaatggtggctccgctttgtttgtggaaggcctggtagtcagtatgctcttgagagtgaaatttggcctgctcccccgatctttattcccactgcccaaccaattaagaatgaatctattgagaaggaattaaagaaactccagaagagatcactcatttctttcacaatcacagggatctctgcaattgcctctgccattggcctcctaatctacttcctgaggcgtaggcgtgaccccgaaccacaggctccgtattctggagctggtggtaggcagcgccagccgccacagcagcggccagtgcctcagaggattgttcattatcaggccccctaccgcatgcctcaaatttaccctaaagtcgagaaaaattgcacctcaatcacttttcaccaggattctcactcttttgatcttacagctctctttatttgtggtagaacttttgtgtgtaataatcatgctttttcccgttcccatacaatcgaaattggaggtcagaaatacaaacctgaagaactttctccagagcttcttgttcgtccctctggcgtgactgatgttgtgatttgtactcttcctcgcggagatgaaagaaagaatcttgttccttatcttctttctcaaaaagaccgaccaacaaatgatgatgtcctcatggtttcaagatcaaagactattgctgctaattttgaatgcacaaacttgagaggaagaaagtctgtgtgcgtgaaggagtttgaaaatgctgatgaacaaaacttccatcgctgttacacctatgacctcaaatcaactcctggtatgtgtggtgctgctctcatttcccgcaaccctgcacgtgaaacccttcttgggattcattttgcagggggtcctggtgttggtattggtgttcctctctacaaagaagattttgcccatctctttcaagggaatctcaaacctattgaacatcccggtcaacccaaccacataccccgtcgctctgctttgaagaaatctcctgcttatggtgcttttcctgtaaagtctgaacctgctatcctttctcaaaaggacaagagatgtgaagtggaccttgatgaggtcatgttcagcaagcatgtccctgaccatgagggctggccaactcttgaaccggctatggcctacgtggttgaggagctaatgcagaaatgtggcttctccaaggatgaccctgttccgatgtggactcttgaacaagcaattaatggtgatggtgtgatggaaggaattgatatggggcagtcggctggataccctttctcagctcaagggcgctcccgtcggagcttctttgaatggaatggcgagaagtggcaagctactgaagaacttaagaagcttgtcgaccatgctctccagcatccagacgactactactatgctacattcctcaaggatgagcttagaccttctgagaaagtgaaagctgggaaaactcgccttgttgattctgattcccttccccgtatccttgcaatgagaatggtgtttggtcccctttttgaagctatgcttaggaaaaatggatcagaaattcactccgctgtgggttgtaatcctgatgttgattggaccaggttttattatgagatgggtcctgattcttttccctactgtttcgatttggactattcctgcttcgattcctctgaacccaaaattgcttttcttctgatggccaagtatcttgaaccctacttccaacatgatgtcacccccttcctctctgcagttgcaacatcgaaacatgtctatggtgataaggcatatgagatggagggaggaatgccttcgggttgtgttgggacaagtatgtttaactgcatcaataattctgctttcattgtttctgctttgattgccttgaagatctcccccgactcttgcaagtggatttgttatggtgatgatgtgattatttctactgatgagaaagctctatcccgtaggattgctgatttctactctaggaataccaatctgaaagttactcctgcttgcaaatctggtgattttcccgaagagtcaaccttctatgaagtatccttcctgaagcgattctttgttcacgactcccactatccccagttgattcatccctacatgccccttgaacatctggaacaatcatccatgtggcaaactgatggcgaatatcaacagaaattggactctctggctcaattggctttccatgctggtggacccgactataggaaattttgtgataccattcagaggaggtgccgctcgcggggcactgaggtttactttaggccttttgagtactacatggctatgtggtacgcacactttatgtaaatcatgggtattaatgtttcaacaattaataatataaaccagatctttgccacaggctcgactgttacatccttcacccttcaatcggcctctatcgtggttatgtgtataggtgctttagtattaataattttagctggtgttgtgatttattatttttggaagaaacatc

SEQ ID NO: 2 - protein, 2813 aa, Artificial Sequence
  Description of Artificial Sequence Synthetic polypeptide
MSYKPEDNAPLQSQQEDNAPSVAPHEKATVETTVTAVIQSEQDIIFDNSWTDVRTIDAEIPRIVATYSWTSSQASGDFLTAVTGSDAVETLPSSPNPFCLPNYLLVNQPSSSTGLNVPAQVGLFQALFTRHALYRSGFRVEVTVNGTQFHGGSLFVVAIPHPHVWIQSLTGSDAGTQKKWTALELVQVNQLGVFPSARLLPRNASAVTLDLPYVGPAPQLETAALDVMYAIAILVETPLSIPTGTAPTLTVVMRVGATNAHFFAPKPYQTPQSNLGAFDVPLDVPAPASLLQGVRVNTAQSAFVNTSSGSVAQLEKRSRNTGTSFLPARIEDWRSVLSRPTLMSLVQFTAATTLGTNIFQANVCPTGTSVTNRDTTQALPSASIKRLQGVPYLAQLARYFSQWRGSLTYTFEYTGPQVSSGRLLIAFLPGRLRAAKSGQGADYMETGALLQNFTENLHVIWDLSNSTSVSITCPYAVATPWAPIVINSWQAIPLSGYSSGTLVAVVMSPLVTPTVVAPTSTILVHLSAGPDFEMRFPAPIPVNTIPMYNGVVDQGPVVESLPQGEHYLTTMDTMSLQTFFDQARFYGNFSATSGTTASMVTIPLSLVTFASSSGTPKTPEVPVRMFAGLFTFLRADLRITVGIPFSQNLIVTYRPPTATPSAYSATGAGAPDSAASLLNGPSVILSTRASNMGVEFVIPFSAFASVFQTSWSQANRVDTNYNPSTNQTNPGDMGTIFVASRLPSAQLSNVSVFIAFQNAEFFVPRPFPPLTSTGWDGTAPNNLEGDFWEDDSTPVEFQSPTSETLEKGDQVMLSVTMDILDSIRELCTELMEGVDTLYGRARAAKFAWMVQSTFEEWKKSDQDADEFFSDVSLVEPVVFQSPPPPSAREKAARLEEFFDYMYGCVESEEELHAVCDAVNAVIDSFYGSDGDFIGEIRDRFSELPLSYPEGPDHPGVWFRGEFYRCLYVTTQNRFFPQVAAYSPVETEHNLFALYEAMRQFELMCIADPLSTSHHYQRLLFLLLQQQWVIPEMKSMIREKLAFLYTMVAHPERIFLDWNWELFHTQPFRYICNMVGVETALTDDGFFSGDEEGQFQAPTSCDQKFLNLVRAYRDAMIVNPWHTFEVCYISFYDLTCRNDVPEWFKRAVEDACFDTLCARVAERVNVNNEVLIERYRQLYDEIFSEEPDFFSDDEIDAEFQAPKPPTPASSVPVPRPSTPLPSPVTSEDEDEGEDEPDDICVYYVDNGLYRHWGIRNKQQAISLKAHGFQCRVSYTPEDLGKHVDYCTVGYNEWCKAVCRLGQIYDYSITHNCTDFVQEVTSYQHSFQNTGYWLAAGVVAAAVATTAIFHSVPPSWDERPFLPEKTTPRVVVVAPHRRMHRWRKATLEKIIELKRKMLFQAPINIQLTNPAVDRAASITTSATLETLDEVKNMVPVLVNSASTVASSLSAASASVEHLTKSMETFLENATSAIPGAVSAMESTAGELISSASKKVASIILKVIGFTLVIFGNPSPSTIAGVIALLVAEVYDNAYLRKKMSDLAVSFTSKIKDLVGSLFGVSSCCDEEPIFTDIDDAFHRYMLTRTETDFEFQAPSRLQEFNQGVLAMKNVEWIIEKIKELIDWLISKLRSKERSSPEDYLKTRADYITDLYRDSLATASCQNVDVNLLNQRVRDTRDMLAYCTNHRLAGAAHILSRTLANYEQTSRKLKTSTYSSRPEPLVIYIHGSPGVGKSLLSNIIASAYCKRKGIDFKTSVFAQPPGSEYFDGYTGQPVHVIDDFCQNTTGEDVKLFCQMVSTTPFVPPMAALEEKGVQYRSRLILATSNMATPQSNEIRIPGALERRCHIKVRAILARAFQTPAGRLDMAEAFKELGPAKSPDFKADCPYLNGAAMTLSVTRGEDRTREHMSVYDLCDMIFDELDERDGNQTAFSKIIFQAPSQNVISFCKHADHSPDSCYNIFIATPQGPIVHRFDSVEDRNSWLQSQIPSLPKAGNVRCCPCKNPDCAKIQFTFKTDDEQIRSQVINFPGRDVAEWWLRFVCGRPGSQYALESEIWPAPPIFIPTAQPIKNESIEKELKKLQKRSLISFTITGISAIASAIGLLIYFLRRRRDPEPQAPYSGAGGRQRQPPQQRPVPQRIVHYQAPYRMPQIYPKVEKNCTSITFHQDSHSFDLTALFICGRTFVCNNHAFSRSHTIEIGGQKYKPEELSPELLVRPSGVTDVVICTLPRGDERKNLVPYLLSQKDRPTNDDVLMVSRSKTIAANFECTNLRGRKSVCVKEFENADEQNFHRCYTYDLKSTPGMCGAALISRNPARETLLGIHFAGGPGVGIGVPLYKEDFVHLFQGNLKPIEHPGQPNHIPRRSALKKSPAYGAFPVKSEPAILSQKDKRCEVDLDEVMFSKHVPDHEGWPTLEPAMAYVVEELMQKCGFSKDDPVPMWTLEQAINGDGVMEGIDMGQSAGYPFSAQGRSRRSFFEWDGEKWQATEELKKLVDHALQHPDDYYYATFLKDELRPSEKVKAGKTRLVDSDSLPRILAMRMVFGPLFEAMLRKNGSEIHSAVGCNPDVDWTRFYYEMGPDSFPYCFDLDYSCFDSSEPKIAFLLMAKYLEPYFQHDVTPFLSAVATSKHVYGDKAYEMEGGMPSGCVGTSMFNCINNSAFIVSALIALKISPDSCKWICYGDDVIISTDEKALSRRIADFYSKNTNLKVTPACKSGDFPEESTFYEVSFLKRFFVHDSHYPQLIHPYMPLEHLEQSSMWQTDGEYQQKLDSLAQLAFHAGGPDYRRFCDTIQKRCRSRGTEVYFRPFEYYMAMWYAHFM

SEQ ID NO: 3 - protein, 798 aa, Artificial Sequence
  Description of Artificial Sequence Synthetic polypeptide
MSYKPEDNAPLQSQQEDNAPSVAPHEKATVETTVTAVIQSEQDIIFDNSWTDVRTIDAEIPRIVATYSWTSSQASGDFLTAVTGSDAVETLPSSPNPFCLPNYLLVNQPSSSTGLNVPAQVGLFQALFTRHALYRSGFRVEVTVNGTQFHGGSLFVVAIPHPHVWIQSLTGSDAGTQKKWTALELVQVNQLGVFPSARLLPRNASAVTLDLPYVGPAPQLETAALDVMYAIAILVETPLSIPTGTAPTLTVVMRVGATNAHFFAPKPYQTPQSNLGAFDVPLDVPAPASLLQGVRVNTAQSAFVNTSSGSVAQLEKRSRNTGTSFLPARIEDWRSVLSRPTLMSLVQFTAATTLGTNIFQANVCPTGTSVTNRDTTQALPSASIKRLQGVPYLAQLARYFSQWRGSLTYTFEYTGPQVSSGRLLIAFLPGRLRAAKSGQGADYMETGALLQNFTENLHVIWDLSNSTSVSITCPYAVATPWAPIVINSWQAIPLSGYSSGTLVAVVMSPLVTPTVVAPTSTILVHLSAGPDFEMRFPAPIPVNTIPMYNGVVDQGPVVESLPQGEHYLTTMDTMSLQTFFDQARFYGNFSATSGTTASMVTIPLSLVTFASSSGTPKTPEVPVRMFAGLFTFLRADLRITVGIPFSQNLIVTYRPPTATPSAYSATGAGAPDSAASLLNGPSVILSTRASNMGVEFVIPFSAFASVFQTSWSQANRVDTNYNPSTNQTNPGDMGTIFVASRLPSAQLSNVSVFIAFQNAEFFVPRPFPPLTSTGWDGTAPNNLEGDFWEDDSTPVEFQ

SEQ ID NO: 4 - protein, 388 aa, Artificial Sequence
  Description of Artificial Sequence Synthetic polypeptide
MSYKPEDNAPLQSQQEDNAPSVAPHEKATVETTVTAVIQSEQDIIFDNSWTDVRTIDAEIPRIVATYSWTSSQASGDFLTAVTGSDAVETLPSSPNPFCLPNYLLVNQPSSSTGLNVPAQVGLFQALFTRHALYRSGFRVEVTVNGTQFHGGSLFVVAIPHPHVWIQSLTGSDAGTQKKWTALELVQVNQLGVFPSARLLPRNASAVTLDLPYVGPAPQLETAALDVMYAIAILVETPLSIPTGTAPTLTVVMRVGATNAHFFAPKPYQTPQSNLGAFDVPLDVPAPASLLQGVRVNTAQSAFVNTSSGSVAQLEKRSRNTGTSFLPARIEDWRSVLSRPTLMSLVQFTAATTLGTNIFQANVCPTGTSVTNRDTTQALPSASIKRLQ

SEQ ID NO: 5 - protein, 166 aa, Artificial Sequence
  Description of Artificial Sequence Synthetic polypeptide
GVPYLAQLARYFSQWRGSLTYTFEYTGPQVSSGRLLIAFLPGRLRAAKSGQGADYMETGALLQNFTENLHVIWDLSNSTSVSITCPYAVATPWAPIVINSWQAIPLSGYSSGTLVAVVMSPLVTPTVVAPTSTILVHLSAGPDFEMRFPAPIPVNTIPMYNGVVDQ

SEQ ID NO: 6 - protein, 244 aa, Artificial Sequence
  Description of Artificial Sequence Synthetic polypeptide
GPVVESLPQGEHYLTTMDTMSLQTFFDQARFYGNFSATSGTTASMVTIPLSLVTFASSSGTPKTPEVPVRMFAGLFTFLRADLRITVGIPFSQNLIVTYRPPTATPSAYSATGAGAPDSAASLLNGPSVILSTRASNMGVEFVIPFSAFASVFQTSWSQANRVDTNYNPSTNQTNPGDMGTIFVASRLPSAQLSNVSVFIAFQNAEFFVPRPFPPLTSTGWDGTAPNNLEGDFWEDDSTPVEFQ

SEQ ID NO: 7 - protein, 82 aa, Artificial Sequence
  Description of Artificial Sequence Synthetic polypeptide
SPTSETLEKGDQVMLSVTMDILDSIRELCTELMEGVDTLYGRARAAKFAWMVQSTFEEWKKSDQDADEFFSDVSLVEPVVFQ

SEQ ID NO: 8 - protein, 319 aa, Artificial Sequence
  Description of Artificial Sequence Synthetic polypeptide
SPPPPSAREKAARLEEFFDYMYGCVESEEELHAVCDAVNAVIDSFYGSDGDFIGEIRDRFSELPLSYPEGPDHPGVWFRGEFYRCLYVTTQNRFFPQVAAYSPVETEHNLFALYEAMRQFELMCIADPLSTSHHYQRLLFLLLQQQWVIPEMKSMIREKLAFLYTMVAHPERIFLDWNWELFHTQPFRYICNMVGVETALTDDGFFSGDEEGQFQAPTSCDQKFLNLVRAYRDAMIVNPWHTFEVCYISFYDLTCRNDVPEWFKRAVEDACFDTLCARVAERVNVNNEVLIERYRQLYDEIFSEEPDFFSDDEIDAEFQ

SEQ ID NO: 9 - protein, 764 aa, Artificial Sequence
  Description of Artificial Sequence Synthetic polypeptide
APKPPTPASSVPVPRPSTPLPSPVTSEDEDEGEDEPDDICVYYVDNGLYRHWGIRNKQQAISLKAHGFQCRVSYTPEDLGKHVDYCTVGYNEWCKAVCRLGQIYDYSITHNCTDFVQEVTSYQHSFQNTGYWLAAGVVAAAVATTAIFHSVPPSWDERPFLPEKTTPRVVVVAPHRRMHRWRKATLEKIIELKRKMLFQAPINIQLTNPAVDRAASITTSATLETLDEVKNMVPVLVNSASTVASSLSAASASVEHLTKSMETFLENATSAIPGAVSAMESTAGELISSASKKVASIILKVIGFTLVIFGNPSPSTIAGVIALLVAEVYDNAYLRKKMSDLAVSFTSKIKDLVGSLFGVSSCCDEEPIFTDIDDAFHRYMLTRTETDFEFQAPSRLQEFNQGVLAMKNVEWIIEKIKELIDWLISKLRSKERSSPEDYLKTRADYITDLYRDSLATASCQNVDVNLLNQRVRDTRDMLAYCTNHRLAGAAHILSRTLANYEQTSRKLKTSTYSSRPEPLVIYIHGSPGVGKSLLSNIIASAYCKRKGIDFKTSVFAQPPGSEYFDGYTGQPVHVIDDFCQNTTGEDVKLFCQMVSTTPFVPPMAALEEKGVQYRSRLILATSNMATPQSNEIRIPGALERRCHIKVRAILARAFQTPAGRLDMAEAFKELGPAKSPDFKADCPYLNGAAMTLSVTRGEDRTREHMSVYDLCDMIFDELDERDGNQTAFSKIIFQAPSQNVISFCKHADHSPDSCYNIFIATPQM

SEQ ID NO: 10 - protein, 199 aa, Artificial Sequence
  Description of Artificial Sequence Synthetic polypeptide
APKPPTPASSVPVPRPSTPLPSPVTSEDEDEGEDEPDDICVYYVDNGLYRHWGIRNKQQAISLKAHGFQCRVSYTPEDLGKHVDYCTVGYNEWCKAVCRLGQIYDYSITHNCTDFVQEVTSYQHSFQNTGYWLAAGVVAAAVATTAIFHSVPPSWDERPFLPEKTTPRVVVVAPHRRMHRWRKATLEKIIELKRKMLFQ

SEQ ID NO: 11 - protein, 192 aa, Artificial Sequence
  Description of Artificial Sequence Synthetic polypeptide
APINIQLTNPAVDRAASITTSATLETLDEVKNMVPVLVNSASTVASSLSAASASVEHLTKSMETFLENATSAIPGAVSAMESTAGELISSASKKVASIILKVIGFTLVIFGNPSPSTIAGVIALLVAEVYDNAYLRKKMSDLAVSFTSKIKDLVGSLFGVSSCCDEEPIFTDIDDAFHRYMLTRTETDFEFQ

SEQ ID NO: 12 - protein, 372 aa, Artificial Sequence
  Description of Artificial Sequence Synthetic polypeptide
APSRLQEFNQGVLAMKNVEWIIEKIKELIDWLISKLRSKERSSPEDYLKTRADYITDLYRDSLATASCQNVDVNLLNQRVRDTRDMLAYCTNHRLAGAAHILSRTLANYEQTSRKLKTSTYSSRPEPLVIYIHGSPGVGKSLLSNIIASAYCKRKGIDFKTSVFAQPPGSEYFDGYTGQPVHVIDDFCQNTTGEDVKLFCQMVSTTPFVPPMAALEEKGVQYRSRLILATSNMATPQSNEIRIPGALERRCHIKVRAILARAFQTPAGRLDMAEAFKELGPAKSPDFKADCPYLNGAAMTLSVTRGEDRTREHMSVYDLCDMIFDELDERDGNQTAFSKIIFQAPSQNVISFCKHADHSPDSCYNIFIATPQ

SEQ ID NO: 13 - protein, 850 aa, Artificial Sequence
  Description of Artificial Sequence Synthetic polypeptide
GPIVHRFDSVEDRNSWLQSQIPSLPKAGNVRCCPCKNPDCAKIQFTFKTDDEQIRSQVINFPGRDVAEWWLRFVCGRPGSQYALESEIWPAPPIFIPTAQPIKNESIEKELKKLQKRSLISFTITGISAIASAIGLLIYFLRRRRDPEPQAPYSGAGGRQRQPPQQRPVPQRIVHYQAPYRMPQIYPKVEKNCTSITFHQDSHSFDLTALFICGRTFVCNNHAFSRSHTIEIGGQKYKPEELSPELLVRPSGVTDVVICTLPRGDERKNLVPYLLSQKDRPTNDDVLMVSRSKTIAANFECTNLRGRKSVCVKEFENADEQNFHRCYTYDLKSTPGMCGAALISRNPARETLLGIHFAGGPGVGIGVPLYKEDFVHLFQGNLKPIEHPGQPNHIPRRSALKKSPAYGAFPVKSEPAILSQKDKRCEVDLDEVMFSKHVPDHEGWPTLEPAMAYVVEELMQKCGFSKDDPVPMWTLEQAINGDGVMEGIDMGQSAGYPFSAQGRSRRSFFEWDGEKWQATEELKKLVDHALQHPDDYYYATFLKDELRPSEKVKAGKTRLVDSDSLPRILAMRMVFGPLFEAMLRKNGSEIHSAVGCNPDVDWTRFYYEMGPDSFPYCFDLDYSCFDSSEPKIAFLLMAKYLEPYFQHDVTPFLSAVATSKHVYGDKAYEMEGGMPSGCVGTSMFNCINNSAFIVSALIALKISPDSCKWICYGDDVIISTDEKALSRRIADFYSKNTNLKVTPACKSGDFPEESTFYEVSFLKRFFVHDSHYPQLIHPYMPLEHLEQSSMWQTDGEYQQKLDSLAQLAFHAGGPDYRRFCDTIQKRCRSRGTEVYFRPFEYYMAMWYAHF

SEQ ID NO: 14 - protein, 150 aa, Artificial Sequence
  Description of Artificial Sequence Synthetic polypeptide
GPIVHRFDSVEDRNSWLQSQIPSLPKAGNVRCCPCKNPDCAKIQFTFKTDDEQIRSQVINFPGRDVAEWWLRFVCGRPGSQYALESEIWPAPPIFIPTAQPIKNESIEKELKKLQKRSLISFTITGISAIASAIGLLIYFLRRRRDPEPQ

SEQ ID NO: 15 - protein, 27 aa, Artificial Sequence
  Description of Artificial Sequence Synthetic peptide
APYSGAGGRQRQPPQQRPVPQRIVHYQ

SEQ ID NO: 16 - protein, 202 aa, Artificial Sequence
  Description of Artificial Sequence Synthetic polypeptide
APYRMPQIYPKVEKNCTSITFHQDSHSFDLTALFICGRTFVCNNHAFSRSHTIEIGGQKYKPEELSPELLVRPSGVTDVVICTLPRGDERKNLVPYLLSQKDRPTNDDVLMVSRSKTIAANFECTNLRGRKSVCVKEFENADEQNFHRCYTYDLKSTPGMCGAALISRNPARETLLGIHFAGGPGVGIGVPLYKEDFVHLFQ

SEQ ID NO: 17 - protein, 472 aa, Artificial Sequence
  Description of Artificial Sequence Synthetic polypeptide
GNLKPIEHPGQPNHIPRRSALKKSPAYGAFPVKSEPAILSQKDKRCEVDLDEVMFSKHVPDHEGWPTLEPAMAYVVEELMQKCGFSKDDPVPMWTLEQAINGDGVMEGIDMGQSAGYPFSAQGRSRRSFFEWDGEKWQATEELKKLVDHALQHPDDYYYATFLKDELRPSEKVKAGKTRLVDSDSLPRILAMRMVFGPLFEAMLRKNGSEIHSAVGCNPDVDWTRFYYEMGPDSFPYCFDLDYSCFDSSEPKIAFLLMAKYLEPYFQHDVTPFLSAVATSKHVYGDKAYEMEGGMPSGCVGTSMFNCINNSAFIVSALIALKISPDSCKWICYGDDVIISTDEKALSRRIADFYSKNTNLKVTPACKSGDFPEESTFYEVSFLKRFFVHDSHYPQLIHPYMPLEHLEQSSMWQTDGEYQQKLDSLAQLAFHAGGPDYRRFCDTIQKRCRSRGTEVYFRPFEYYMAMWYAHFM

SEQ ID NO: 18 - DNA, 19 bp, Artificial Sequence
  Description of Artificial Sequence Synthetic primer
caccctctaygggcaatgt

SEQ ID NO: 19 - DNA, 20 bp, Artificial Sequence
  Description of Artificial Sequence Synthetic primer
tcagccagtctatggccagg

SEQ ID NO: 20 - DNA, 24 bp, Artificial Sequence
  Description of Artificial Sequence Synthetic probe
tggattcccatctcacgcgtccac

SEQ ID NO: 21 - DNA, 20 bp, Artificial Sequence
  Description of Artificial Sequence Synthetic primer
caccctyyaygggcaaatgt

SEQ ID NO: 22 - DNA, 21 bp, Artificial Sequence
  Description of Artificial Sequence Synthetic probe
attcccatctcacgcgtccac

SEQ ID NO: 23 - protein, 4 aa, Artificial Sequence
  Description of Artificial Sequence Synthetic peptide
HWGI

SEQ ID NO: 24 - protein, 8 aa, Artificial Sequence
  Description of Artificial Sequence Synthetic peptide
GSPGVGKS

SEQ ID NO: 25 - protein, 5 aa, Artificial Sequence
  Description of Artificial Sequence Synthetic peptide
GMCGA

SEQ ID NO: 26 - protein, 5 aa, Artificial Sequence
  Description of Artificial Sequence Synthetic peptide
GDFLT

SEQ ID NO: 27 - protein, 9 aa, Artificial Sequence
  Description of Artificial Sequence Synthetic peptide
EVSFLKRFF

SEQ ID NO: 28 - protein, 12 aa, Artificial Sequence
  Description of Artificial Sequence Synthetic peptide
TNLKVTPACKSG

SEQ ID NO: 29 - protein, 10 aa, Artificial Sequence
  Description of Artificial Sequence Synthetic peptide
ICYGDDVIIS

SEQ ID NO: 30 - protein, 22 aa, Artificial Sequence
  Description of Artificial Sequence Synthetic peptide
YEMEGGMPSGCVGTSMFNCINN

SEQ ID NO: 31 - protein, 16 aa, Artificial Sequence
  Description of Artificial Sequence Synthetic peptide
SFPYCFDLDYSCFDSS

(19) United States
(12) Patent Application Publication
(10) Pub. No.: US 2013/0071421 A1
(43) Pub. Date: Mar. 21, 2013

(54) TURKEY VIRAL HEPATITIS VIRUS AND USES THEREOF

(71) Applicants: W. Ian Lipkin, New York, NY (US); Thomas Briese, White plains, NY (US); Kirsi Honkavuori, New York, NY (US); H.L. Shivaprasad, Davis, CA (US)

(72) Inventors: W. Ian Lipkin, New York, NY (US); Thomas Briese, White plains, NY (US); Kirsi Honkavuori, New York, NY (US); H.L. Shivaprasad, Davis, CA (US)

(21) Appl. No.: 13/499,871

(22) PCT Filed: Oct. 4, 2010
(86) PCT No.: PCT/US10/51345

§ 371 Date: Nov. 21, 2012

Related U.S. Application Data
(60) Provisional application No. 61/248,078, filed on Oct. 2, 2009.

(51) Int. Cl. A61K 39/29; G01N 33/576; A61K 31/7088; C12Q 1/70

(52) U.S. Cl. CPC A61K 39/29; C12Q 1/707; G01N 33/5767; A61K 31/7088
USPC 424/189.1; 536/23.72; 536/24.32; 435/5; 536/24.5; 435/235.1; 435/236; 530/350; 514/44 A; 530/387.9; 530/387.3; 530/324

(57) ABSTRACT

Isolated a Turkey Viral Hepatitis picomavirus-like viruses associated with Turkey viral hepatitis, and isolated nucleic acid sequences and polypeptides are disclosed. Antibodies against antigens from Turkey Viral Hepatitis picornavirus-like viruses, iRNAs which target nucleic acid sequences of the Turkey Viral Hepatitis picornavirus-like virus, methods for detecting the presence or absence of Turkey Viral Hepatitis picomavirus-like viruses in an animal, and immunogenic compositions for inducing an immune response against Turkey Viral Hepatitis picomavirus-like viruses in an animal are also disclosed.

This application claims the benefit of and priority to U.S. provisional patent application Ser. No. 61/248,078, filed Oct. 2, 2009, the disclosure of which are hereby incorporated by reference in their entireties for all purposes.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

Turkey viral hepatitis (TVH) is a highly infectious disease affecting young turkey poults. The disease is often subclinical with minor histological lesions, becoming apparent when the animals are stressed, resulting in varying rates of morbidity and mortality (Klein et al, Avian Dis. 1991; 35:115-25). TVH is an acute, highly contagious and often subclinical disease of turkey poults 5 wk of age. The disease is widespread, with morbidity rates up to 100% and mortality, which can be restricted to poults, up to 25%. The disease often comes apparent when the birds are stressed. This may result in reduction in the production, fertility and hatchability in the flocks.

Clinical signs of TVH include anorexia, depression and loose fecal matter. A diagnosis is made on the basis of characteristic lesions in the liver that include multifocal necrosis and mononuclear inflammatory cell infiltrates (Mongeau et al, Avian Dis. 1959; 3:388-96; Snoeyenbos et al, Avian Dis. 1959; 3:377-88). Similar lesions may be found in the pancreas. Mortality rates in poults reach up to 25% (Snoeyenbos et al, Avian Dis. 1960; 3:477-84).

The disease has been experimentally reproduced in turkey poults by inoculation with material derived from affected animals (Klein et al, Avian Dis. 1991; 35:115-25; Mongeau et al, Avian Dis. 1959; 3:388-96; Snoeyenbos et al, Avian Dis. 1959; 3:377-88; Snoeyenbos et al, Avian Dis. 1960; 3:477-84). A viral basis for TVH has been presumed since its initial description in 1959, because the causative agent passed through 100 nm membranes, was acid stable, not affected by antibiotics and could be propagated in the yolk sac of embryonated chicken eggs (Mongeau et al, Avian Dis. 1959; 3:388-96; Snoeyenbos et al, Avian Dis. 1959; 3:377-88; Tzianabos et al, Avian Dis. 1965; 9:152-6). Icosahedral particles of 24-30 nm size have been found by electron microscopy (EM) in liver lesions of birds (MacDonald et al., Vet. Rec. 1982; 111:323) as well as embryonated turkey eggs (Klein et al, Avian Dis. 1991; 35:115-25) inoculated with material derived from affected birds; however, no agent has been consistently implicated (Cho, Avian Dis. 1976; 20:714-23; Wages and Ficken. Avian Dis. 1989; 33:191-4).

There is a need for a diagnostic test, a vaccine or and a method of treating animals having this economically important poultry disease. This invention addresses these needs.

### SUMMARY OF THE INVENTION

In one aspect, the invention provides an isolated nucleic acid having a sequence of SEQ ID NO: 1. In another aspect, the invention provides an isolated nucleic acid which comprises 10 consecutive nucleotides having a sequence of SEQ ID NO: 1.

In still a further aspect, the invention provides an isolated nucleic acid which is a variant of any one of SEQ ID NO: 1 and has at least about 85% identity to SEQ ID NO: 1. In one embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1. In another embodiment, the identity is determined by analysis with a sequence comparison algorithm.

In yet another aspect, the invention relates to an isolated nucleic acid complementary to a sequence of SEQ ID NO: 1. In still a further aspect, the invention provides an isolated nucleic acid which comprises 10 consecutive nucleotides complementary to a sequence of SEQ ID NO: 1. In yet another aspect, the invention provides an isolated nucleic acid which is a complementary to a variant of any one of SEQ ID NO: 1 and wherein the variant has at least about 85% identity to SEQ ID NO: 1. In one embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1. In another embodiment, the identity is determined by analysis with a sequence comparison algorithm. In yet another embodiment, the sequence comparison algorithm is FASTA version 3.0t78 using default parameters.

In still a further aspect, the invention provides an isolated polypeptide having a sequence selected from the group consisting of: SEQ ID NOs: 2-17.

In yet another aspect, the invention provides an isolated polypeptide which comprises 8 consecutive amino acids having a sequence selected from the group consisting of: SEQ ID NOs: 2-17.

In still a further aspect, the invention provides an isolated polypeptide which is a variant of any one of SEQ ID NOs: 2-17 and has at least about 85% identity to SEQ ID NO: 2-17. In one embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 2-17. In another embodiment, the identity of the isolated polypeptide is determined by analysis with a sequence comparison algorithm. In yet another embodiment, the sequence comparison algorithm is FASTA version 3.0t78 using default parameters.

In still a further aspect, the invention provides an oligonucleotide probe which comprises from about 10 nucleotides to about 50 nucleotides, wherein at least about 10 contiguous nucleotides are at least 95% complementary to a nucleic acid target region within a nucleic acid sequence of SEQ ID NO: 1. In one embodiment, the probe is at least about 95.5%, about 96%, about 96.5%, about 97%, about 97.5%, about 98%, about 98.5%, about 99%, about 99.5% or about 99.9% complementary to SEQ ID NO: 1. In still another embodiment, the oligonucleotide probe consists essentially of from about 10 to about 50 nucleotides.

In yet another aspect, the invention provides a synthetic nucleic acid which has a sequence consisting of from about 10 to about 30 consecutive nucleotides from a nucleic acid sequence of SEQ ID NO: 1.

In still a further aspect, the invention provides a method for determining the presence or absence of TVHV in a biological sample, the method comprising: a) contacting nucleic acid from a biological sample with at least one primer which is an oligonucleotide probe which comprises from about 10 nucleotides to about 50 nucleotides, wherein at least about 10 contiguous nucleotides are at least 95% complementary to a nucleic acid target region within a nucleic acid sequence of SEQ ID NO: 1, b) subjecting the nucleic acid and the primer to amplification conditions, and c) determining the presence or absence of amplification product, wherein the presence of amplification product indicates the presence of RNA associated with TVHV in the sample.

In yet another aspect, the invention provides a synthetic nucleic acid which has a sequence consisting of from about 10 to about 30 consecutive nucleotides from a nucleic acid sequence which is complementary to a nucleic acid sequence of SEQ ID NO: 1.

In yet another aspect, the invention provides a method for determining the presence or absence of TVHV in a biological sample, the method comprising: a) contacting nucleic acid from a biological sample with at least one primer which is an oligonucleotide probe which comprises from about 10 nucleotides to about 50 nucleotides, wherein at least about 10 contiguous nucleotides are at least 95% complementary to a nucleic acid target region within a nucleic acid sequence of SEQ ID NO: 1, and b) subjecting the nucleic acid and the primer to amplification conditions, and c) determining the presence or absence of amplification product, wherein the presence of amplification product indicates the presence of RNA associated with TVHV in the sample.

In another aspect, the invention provides a primer set for determining the presence or absence of TVHV in a biological sample, wherein the primer set comprises at least one synthetic nucleic acid sequence selected from the group consisting of: a) a synthetic nucleic acid which has a sequence consisting of from about 10 to about 30 consecutive nucleotides from a nucleic acid sequence of SEQ ID NO: 1, and b) a synthetic nucleic acid which has a sequence consisting of from about 10 to about 30 consecutive nucleotides from a nucleic acids sequence which is complementary to a nucleic acid sequence of SEQ ID NO: 1.

In yet another aspect, the invention provides a method for determining whether or not a sample contains TVHV, the method comprising: a) contacting a biological sample with an antibody that specifically binds a polypeptide encoded by the nucleic sequence acid of any one of SEQ ID NO: 1, and b) determining whether or not the antibody binds to an antigen in the biological sample, wherein binding indicates that the biological sample contains TVHV. In one embodiment, the determining comprises use of a lateral flow assay or ELISA.

In still another aspect, the invention provides a method for determining whether or not a biological sample has been infected by TVHV, the method comprising: a) determining whether or not a biological sample contains antibody that specifically binds a polypeptide encoded by the nucleic sequence acid of any one of SEQ ID NO: 1.

In yet another aspect, the invention provides an interfering RNA (iRNA) comprising a sense strand having at least 15 contiguous nucleotides complementary to the anti-sense strand of a gene from a virus comprising a nucleic acid sequence of SEQ ID NO: 1.

In still another aspect, the invention provides an interfering RNA (iRNA) comprising an anti-sense strand having at least 15 contiguous nucleotides complementary to the sense strand of gene from a virus comprising a nucleic acid sequence of SEQ ID NO: 1.

In still a further aspect, the invention provides a method for reducing the levels of a viral protein, viral mRNA or viral titer in a cell in an animal comprising: administering an iRNA agent to an animal, wherein the iRNA agent comprises a sense strand having at least 15 contiguous nucleotides complementary to gene from a TVHV comprising a nucleic acid sequence of SEQ ID NO: 1 and an antisense strand having at least 15 contiguous nucleotides complementary to the sense strand. In one embodiment, the method further comprises co-administering a second iRNA agent to the animal, wherein the second iRNA agent comprises a sense strand having at least 15 or more contiguous nucleotides complementary to second gene from the TVHV comprising a nucleic acid sequence of SEQ ID NO: 1 and an antisense strand having at least 15 or more contiguous nucleotides complementary to the sense strand.

In yet another embodiment, the invention provides a method of reducing the levels of a viral protein from at least one gene of a TVHV in a cell in an animal, the method comprising administering an iRNA agent to an animal, wherein the iRNA agent comprises a sense strand having at least 15 or more contiguous from a nucleic acid sequence of SEQ ID NO: 1 complementary to a gene from a TVHV and an antisense strand having at least 15 or more contiguous nucleotides complementary to the sense strand of a nucleic acid sequence of SEQ ID NO: 1.

In certain embodiments, the samples described herein are from an avian. In certain embodiments, the samples described herein are from a turkey

In yet another aspect, the invention provides an isolated virus comprising any one of the nucleic acid sequences of SEQ ID NO: 1.

In still another aspect, the invention provides an isolated virus comprising a polypeptide encoded by the nucleic sequence acid of any one of SEQ ID NO: 1.

In another aspect, the invention provides a TVHV immunogenic composition comprising a TVHV nucleic acid. In one embodiment, the TVHV nucleic acid is a nucleic acid sequence of SEQ ID NO: 1. In still a further embodiment, the TVHV nucleic acid comprises least 24 consecutive nucleic acids of SEQ ID NO: 1. In yet another embodiment, the TVHV nucleic acid is substantially identical to the nucleic acid sequence of SEQ ID NO: 1. In still a further embodiment, the TVHV nucleic acid is a variant of SEQ ID NO: 1 having at least about 85% identity to SEQ ID NO: 1. In one embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1.

In another aspect, the invention provides a TVHV immunogenic composition comprising a TVHV polypeptide. In one embodiment, the TVHV polypeptide is a polypeptide encoded by any of SEQ ID NO: 1. In another embodiment, the TVHV polypeptide is a polypeptide encoded by a nucleic acid comprising least 24 consecutive nucleic acids of SEQ ID NO: 1. In still a further embodiment, the TVHV polypeptide is a polypeptide encoded by a nucleic acid that is substantially identical to the nucleic acid sequence of SEQ ID NO: 1. In another embodiment, the TVHV polypeptide is a polypeptide encoded by a nucleic acid that is a variant of SEQ ID NO: 1 having at least about 85% identity to SEQ ID NO: 1. In still a further embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1. In still another embodiment, the TVHV polypeptide is a polypeptide comprising the amino acid sequence of SEQ ID NOs: 2-17. In yet another embodiment, the TVHV polypeptide is a polypeptide comprising least 8 consecutive amino acids of any of SEQ ID NOs: 2-17. In yet another embodiment, the TVHV polypeptide is substantially identical to the amino acid sequence of any of SEQ ID NOs: 2-17. In yet another embodiment, the TVHV polypeptide is a variant of any of SEQ ID NOs: 2-17 and having at least about 85% identity to SEQ ID NOs: 2-17. In another embodiment, the variant has at least about 90%, about 95.5%, about 96%, about 96.5%, about 97%, about 97.5%, about 98%, about 98.5%, about 99%, about 99.5% or about 99.9% identity to that of any one of SEQ ID NOs: 2-17.

In another aspect, the invention provides an isolated antibody that specifically binds to a polypeptide encoded by the nucleotide sequence shown in any one of SEQ ID NO: 1. In still another aspect, the invention provides an isolated antibody that specifically binds to a polypeptide having the sequence of any of SEQ ID NO: 2-17. In one embodiment, the antibody binds a TVHV or a TVHV polypeptide and inhibits, neutralizes or reduces the function or activity of the TVHV or TVHV polypeptide. In another embodiment, the antibody is a polyclonal antibody. In another embodiment, the antibody is a monoclonal antibody. In another embodiment, the antibody is an avian antibody. In another embodiment, the antibody is a turkey antibody. In another embodiment, the antibody is an IgM antibody. In another embodiment, the antibody is a chimeric antibody.

In still a further aspect, the invention provides an immunogenic composition comprising a killed virus comprising a TVHV polypeptide. In another aspect, the invention provides an immunogenic composition comprising an attenuated virus comprising a TVHV polypeptide. In one embodiment, the immunogenic compositions described herein further comprise at least one excipient, additive or adjuvant.

In another aspect, the invention relates to a method of inducing an immune response in an animal, the method comprising administering the TVHV immunogenic compositions described herein.

In another aspect, the invention provides a method for preventing, or reducing TVHV infection in an animal, the method comprising administering to the animal any of the TVHV immunogenic compositions described herein.

In another aspect, the invention provides a method for preventing, or reducing TVHV infection in an animal, the method comprising administering to the animal any of the antibodies described herein.

In one embodiment, the administration in any of the methods described herein is oral administration or injection administration.

In still another aspect, the invention provides the use of any of the immunogenic compositions described herein in the manufacture of a vaccine for the treatment of condition TVHV infection in an animal.

### BRIEF DESCRIPTION OF THE FIGURES

FIG. 1. Predicted TVHV genome organization based on sequence comparison to known picornaviruses. Dashed lines above the genome depict the location of the original sequences obtained by high throughput sequence analysis. Conserved picornaviral motifs and predicted potential cleavage sites along the coding region are indicated in the bar below.

FIG. 2. Relationships between TVHV and other picornaviruses. The phylogenetic analyses were based on amino acid sequences of the combined 2C, 3C and 3D regions (FIG. 2A), the P1 region (FIG. 2B), and complete coding regions, excluding divergent aa 799-1199 (FIG. 2C). Representative sequences from different picornavirus genera and recently discovered, unclassified viruses were obtained from GenBank with their accession numbers indicated. Bootstrap values are given at the respective nodes; scale bar indicates number of amino acid substitutions per site. TMEV: theiler's murine encephalomyelitis virus, EMCV: encephalomyocarditis virus, SVV: Seneca Valley virus, HCoSV A1: human cosavirus A1, HCoSV D1: human cosavirus D1, ERBV: equine rhinitis B virus, ERAV: equine rhinitis A virus, BRBV: bovine rhinitis B virus, FMDV: foot-and-mouth disease virus, PTV: porcine teschovirus, PEV-8: porcine enterovirus type 8, HRV-C: human rhinovirus C, CV-A21: coxsackievirus A21, EV-96: enterovirus 96, AEV: avian encephalomyelitis-like virus, HAV: hepatitis A virus, SePV: seal picornavirus, DPV: duck picornavirus, HPeV; human parechovirus, LV: Ljungan virus.

FIG. 3. In situ hybridization experiments using TVHV-specific oligonucleotides. Hybridization of β-actin (FIG. 3A) and TVHV-specific probes (FIG. 3B) with FastRed staining on hepatitis-affected liver tissue from poult 2993A and on non-diseased liver tissue from poult 1927B (FIGS. 3C and 3D, respectively). Bright-field microscopy images were acquired at x40 magnification.

FIG. 4. Immunohistologic staining of liver tissues with serum from a TVH affected poult. Serum from PCR-positive poult 394.9 demonstrates TVHV antigens in clusters of cells in liver tissue of TVH-affected poult 2993A (FIG. 4A), but not in liver sections from TVH-negative poult 1927B (FIG. 4B). Sections were counterstained with hematoxylin and bright field images were acquired at ×40 magnification.

FIG. 5. FIG. 5A-E shows a nucleic acid sequence (SEQ ID: NO 1) which is derived from a TVHV.

FIG. 6. FIG. 6A-B shows an amino acid sequence (SEQ ID: NO 2) which is derived from a TVHV.

FIG. 7 shows an amino acid sequence (SEQ ID: NO 3) which is derived from a TVHV.

FIG. 8 shows an amino acid sequence (SEQ ID: NO 4) which is derived from a TVHV.

FIG. 9 shows an amino acid sequence (SEQ ID: NO 5) which is derived from a TVHV.

FIG. 10 shows an amino acid sequence (SEQ ID: NO 6) which is derived from a TVHV.

FIG. 11 shows an amino acid sequence (SEQ ID: NO 7) which is derived from a TVHV.

FIG. 12 shows an amino acid sequence (SEQ ID: NO 8) which is derived from a TVHV.

FIG. 13 shows an amino acid sequence (SEQ ID: NO 9) which is derived from a TVHV.

FIG. 14 shows an amino acid sequence (SEQ ID: NO 10) which is derived from a TVHV.

FIG. 15 shows an amino acid sequence (SEQ ID: NO 11) which is derived from a TVHV.

FIG. 16 shows an amino acid sequence (SEQ ID: NO 12) which is derived from a TVHV.

FIG. 17 shows an amino acid sequence (SEQ ID: NO 13) which is derived from a TVHV.

FIG. 18 shows an amino acid sequence (SEQ ID: NO 14) which is derived from a TVHV.

FIG. 19 shows an amino acid sequence (SEQ ID: NO 15) which is derived from a TVHV.

FIG. 20 shows an amino acid sequence (SEQ ID: NO 16) which is derived from a TVHV.

FIG. 21 shows an amino acid sequence (SEQ ID: NO 17) which is derived from a TVHV.

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, “Turkey Viral Hepatitis picornavirus-like virus” refers to isolates of the Turkey Viral Hepatitis picornavirus-like viruses described herein.

As used herein, the term “animal” refers to a vertebrate, including, but not limited to avians (e.g. turkeys).

The cause for Turkey viral hepatitis disease is unknown. Icosahedral viral particles in affected birds have been described in literature (Klein et al., 1991, Avian Diseases 35: 115-125) but no sequence information nor characterization of the type of virus present is available. In one aspect, the invention relates to the identification of a novel virus displaying limited sequence similarity at amino acid level to picornaviruses in the livers of turkeys suffering from hepatitis.

The present invention provides Turkey Viral Hepatitis picornavirus-like virus nucleic acid sequences.

Picornaviridae, a family of small nonenveloped viruses with positive-sense single strand RNA genome, currently consists of twelve genera. Recent additions to the family include salivirus NG-J1 and human klasseviruses identified in pediatric stool samples (Greninger et al., Virol J. 2009; 6:82; Holtz et al., Virol J. 2009; 6:86; Li et al., J. Virol. 2009; 83:12002-6), cosaviruses (Kapoor et al., Proc Natl Acad Sci USA. 2008; 105:20482-7; Holtz et al., Virol J. 2008; 5:159), and an unclassified seal picornavirus 1 (Kapoor et al., J. Virol. 2008; 82:311-20).

These nucleic acid sequences may be useful for, inter alia, expression of Turkey Viral Hepatitis picornavirus-like virus-encoded proteins or fragments, variants, or derivatives thereof, generation of antibodies against Turkey Viral Hepatitis picornavirus-like virus proteins, generation of primers and probes for detecting Turkey Viral Hepatitis picornavirus-like virus and/or for diagnosing Turkey Viral Hepatitis picornavirus-like virus infection, generating vaccines against Turkey Viral Hepatitis picornavirus-like viruses, and screening for drugs effective against Turkey Viral Hepatitis picornavirus-like viruses as described herein.

In certain aspects, the invention is directed to a Turkey Viral Hepatitis picornavirus-like virus isolated nucleic acid sequence as provided in any one of SEQ ID NO: 1. The Turkey Viral Hepatitis picornavirus-like virus nucleic acids sequences as provided in any one of SEQ ID NO: 1 were identified by extraction and random DNA amplification of RNA from four infected turkey livers. The samples were pooled together and processed by high throughput pyrosequencing.

In one aspect, the present invention shows that Turkey Viral Hepatitis is associated with infection with a novel picornavirus-like virus termed Turkey Viral Hepatitis picornavirus-like virus (TVHV).

In other aspects, the invention is directed to expression constructs, for example plasmids and vectors, and isolated nucleic acids which comprise TVHV nucleic acid sequence of SEQ ID NO: 1, fragments, complementary sequences, and/or variants thereof.

The nucleic acid sequences and polypeptides described herein may be useful for multiple applications, including, but not limited to, generation of antibodies and generation of immunogenic compositions.

For example, in one aspect, the invention is directed to an immunogenic composition comprising a polypeptide encoded by a TVHV nucleic sequence acid of any one of SEQ ID NO: 1.

In another aspect, the invention is directed to an immunogenic composition comprising a polypeptide comprising the amino acid sequence of any one of SEQ ID NOs: 2-17.

In one aspect, the invention provides an isolated TVHV nucleic acid having the sequence of SEQ ID NO: 1, or a fragment thereof.

In another aspect, the invention provides an isolated TVHV nucleic acid which comprises consecutive nucleotides having a sequence of SEQ ID NO: 1, or a fragment thereof.

In another aspect, the invention provides an isolated TVHV nucleic acid which comprises consecutive nucleotides having a sequence selected from a variant of SEQ ID NO: 1 or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In one aspect, the invention provides an isolated TVHV nucleic acid complementary to a TVHV nucleic acid sequence in any of SEQ ID NO: 1, or a fragment thereof.

In another aspect, the invention provides an isolated TVHV nucleic acid which comprises consecutive nucleotides complementary to a TVHV nucleic acid sequence in any of SEQ ID NO: 1, or a fragment thereof.

In another aspect, the invention provides an isolated TVHV nucleic acid which comprises consecutive nucleotides complementary to a TVHV nucleic acid sequence in any of SEQ ID NO: 1, or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In another aspect, the invention provides an isolated TVHV nucleic acid having a sequence substantially identical to a TVHV nucleic acid sequence in any of SEQ ID NO: 1, or a fragment thereof.

In another aspect, the invention provides an isolated TVHV nucleic acid having a sequence substantially identical to a sequence complementary to a TVHV nucleic acid sequence in any of SEQ ID NO: 1, or a fragment thereof.

The TVHV nucleic acid sequences described herein may be useful for, inter alia, expression of TVHV-encoded proteins or fragments, variants, or derivatives thereof, generation of antibodies against TVHV proteins, generating vaccines against TVHV, and screening for drugs effective against TVHV as described herein.

In another aspect, the invention is directed to a polypeptide comprising the amino acid sequence of any one of SEQ ID NOs: 2-17.

In one aspect, the invention provides an isolated TVHV nucleic acid having the sequence of SEQ ID NO: 1, or a fragment thereof.

In another aspect, the invention provides an isolated TVHV nucleic acid which comprises consecutive nucleotides having a sequence of SEQ ID NO: 1, or a fragment thereof.

In another aspect, the invention provides an isolated TVHV nucleic acid which comprises consecutive nucleotides having a sequence selected from a variant of SEQ ID NO: 1 or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In one aspect, the invention provides an isolated TVHV nucleic acid complementary to a TVHV nucleic acid sequence in any of SEQ ID NO: 1, or a fragment thereof.

In another aspect, the invention provides an isolated TVHV nucleic acid which comprises consecutive nucleotides complementary to a TVHV nucleic acid sequence in any of SEQ ID NO: 1, or a fragment thereof.

In another aspect, the invention provides an isolated TVHV nucleic acid which comprises consecutive nucleotides complementary to a TVHV nucleic acid sequence in any of SEQ ID NO: 1, or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In another aspect, the invention provides an isolated TVHV nucleic acid having a sequence substantially identical to a TVHV nucleic acid sequence in any of SEQ ID NO: 1, or a fragment thereof.

In another aspect, the invention provides an isolated TVHV nucleic acid having a sequence substantially identical to a sequence complementary to a TVHV nucleic acid sequence in any of SEQ ID NO: 1, or a fragment thereof.

In another aspect, the invention provides an oligonucleotide probe which comprises from about 10 nucleotides to about 50 nucleotides, wherein at least about 10 contiguous nucleotides are at least 95% complementary to a nucleic acid target region within a TVHV nucleic acid sequence in any of SEQ ID NO: 1, wherein the oligonucleotide probe hybridizes to the nucleic acid target region under moderate to highly stringent conditions to form a detectable nucleic acid target:oligonucleotide probe duplex. In one embodiment, the oligonucleotide probe is at least about 95.5%, about 96%, about 96.5%, about 97%, about 97.5%, about 98%, about 98.5%, about 99%, about 99.5% or about 99.9% complementary to SEQ ID NO: 1. In another embodiment the oligonucleotide probe consists essentially of from about 10 to about 50 nucleotides.

Polynucleotides homologous to the sequences illustrated in the SEQ ID NO: 1 can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations.

In certain aspects, the invention is directed to primer sets comprising isolated nucleic acids as described herein, which primer set are suitable for amplification of nucleic acids from samples which comprises TVHV represented by any one of SEQ ID NO: 1, or variants thereof. Primer sets can comprise any suitable combination of primers which would allow amplification of a target nucleic acid sequences in a sample which comprises TVHV represented by any one of SEQ ID NO: 1, or variants thereof. Amplification can be performed by any suitable method known in the art, for example but not limited to PCR, RT-PCR, transcription mediated amplification (TMA).

Hybridization conditions: As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y. (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-11.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HCl (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)−0.62(% formamide)−500/L (1) DNA-RNA: Tm(° C.)=79.8+18.5(log [Na+])+0.58(% G+C)+0.12(% G+C)²−0.5(% formamide)−820/L (2) RNA-RNA: Tm(C)=79.8+18.5(log [Na+])+0.58(% G+C)+0.12(% G+C)²−0.35(% formamide)−820/L (3), where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency. As a general guidelines high stringency is typically performed at Tm −5° C. to −Tm −20° C., moderate stringency at Tm−20° C. to Tm−35° C. and low stringency at Tm−35° SC to Tm−50° C. for duplex>150 base pairs. Hybridization may be performed at low to moderate stringency (25-50° C. below Tm), followed by post-hybridization washes at increasing stringencies. Maximum rates of hybridization in solution are determined empirically to occur at Tm−25° C. for DNA-DNA duplex and Tm −15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, an animal nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, In Methods in Enzymology: 152: 467-469; and Anderson and Young (1985) “Quantitative Filter Hybridisation.” In: Hames and Higgins, ed., Nucleic Acid Hybridisation, A Practical Approach. Oxford, IRL Press, 73-111.

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from any one of SEQ ID NO: 1, or sequences complementary to any one of SEQ ID NO: 1. The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from any one of SEQ ID NO: 1, or sequences complementary to any one of SEQ ID NO: 1. The invention is also directed to primer and/or probes which can be labeled by any suitable molecule and/or label known in the art, for example but not limited to fluorescent tags suitable for use in Real Time PCR amplification, for example TaqMan, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In yet a further aspect, the invention provides a synthetic nucleic acid which has a sequence consisting of from about 10 to about 30 consecutive nucleotides from a nucleic acid sequence of SEQ ID NO: 1.

In yet a further aspect, the invention provides a synthetic nucleic acid which has a sequence consisting of from about 10 to about 30 consecutive nucleotides from a nucleic acid consisting of consecutive nucleotides having a sequence which is a variant of any one of SEQ ID NO: 1 having at least about 95% identity to SEQ ID NO: 1. In one embodiment, the variant has at least about 95.5%, about 96%, about 96.5%, about 97%, about 97.5%, about 98%, about 98.5%, about 99%, about 99.5% or about 99.9% identity to SEQ ID NO: 1.

In another aspect, the invention provides a composition comprising one or more nucleic acids having a sequence consisting of from about 10 to about 30 consecutive nucleotides from a nucleic acids sequence of SEQ ID NO: 1.

In another aspect, the invention provides a composition comprising one or more nucleic acids having a sequence consisting of from about 10 to about 30 consecutive nucleotides from a nucleic acid consisting of consecutive nucleotides having a sequence which is a variant of any one of SEQ ID NO: 1 having at least about 95% identity to SEQ ID NO: 1. In one embodiment, the variant has at least about 95.5%, about 96%, about 96.5%, about 97%, about 97.5%, about 98%, about 98.5%, about 99%, about 99.5% or about 99.9% identity to SEQ ID NO: 1.

In yet another aspect, the invention provides a synthetic nucleic acid which has a sequence consisting of from about 10 to about 30 consecutive nucleotides from a nucleic acid sequence which is complementary to a nucleic acid sequence of SEQ ID NO: 1.

In yet another aspect, the invention provides a synthetic nucleic acid which has a sequence consisting of from about 10 to about 30 consecutive nucleotides complementary to a nucleic acid consisting of consecutive nucleotides having a sequence which is a variant of any one of SEQ ID NO: 1 having at least about 95% identity to SEQ ID NO: 1. In one embodiment, the variant has at least about 95.5%, about 96%, about 96.5%, about 97%, about 97.5%, about 98%, about 98.5%, about 99%, about 99.5% or about 99.9% identity to SEQ ID NO: 1.

In yet another aspect, the invention a composition comprising one or more synthetic nucleic acids which have a sequence consisting of from about 10 to about 30 consecutive nucleotides from a nucleic acid sequence which is complementary to a nucleic acid sequence of SEQ ID NO: 1.

In yet another aspect, the invention provides a composition comprising one or more synthetic nucleic acids which have a sequence consisting of from about 10 to about 30 consecutive nucleotides complementary to a nucleic acid consisting of consecutive nucleotides having a sequence which is a variant of any one of SEQ ID NO: 1 having at least about 95% identity to SEQ ID NO: 1. In one embodiment, the variant has at least about 95.5%, about 96%, about 96.5%, about 97%, about 97.5%, about 98%, about 98.5%, about 99%, about 99.5% or about 99.9% identity to SEQ ID NO: 1.

In other aspects the invention is directed to isolated nucleic acid sequences such as primers and probes, comprising nucleic acid sequences derived from any one of SEQ ID NO: 1. Such primers and/or probes may be useful for detecting the presence of the TVHV of the invention, for example in samples of bodily fluids such as blood, saliva, or urine from an animal, and thus may be useful in the diagnosis of TVHV infection. Such probes can detect polynucleotides of SEQ ID NO: 1 in samples which comprise TVHV represented by SEQ ID NO: 1. The isolated nucleic acids which can be used as primer and/probes are of sufficient length to allow hybridization with, i.e. formation of duplex with a corresponding target nucleic acid sequence, a nucleic acid sequences of any one of SEQ ID NO: 1, or a variant thereof.

In one embodiment, the invention is directed to an isolated nucleic acid sequence comprising from about 10 to about 50 consecutive nucleotides from any one of SEQ ID NO: 1 or a sequence complementary to any one of SEQ ID NO: 1. In one embodiment, the invention is directed to an isolated nucleic acid sequence comprising from about 10 to about 100 consecutive nucleotides from any one of SEQ ID NO: 1 or a sequence complementary to any one of SEQ ID NO: 1. In one embodiment, the invention is directed to an isolated nucleic acid sequence comprising from about 10 to about 200 consecutive nucleotides from any one of SEQ ID NO: 1 or a sequence complementary to any one of SEQ ID NO: 1. In one embodiment, the invention is directed to an isolated nucleic acid sequence comprising from about 10 to about 300 consecutive nucleotides from any one of SEQ ID NO: 1 or a sequence complementary to any one of SEQ ID NO: 1. In one embodiment, the invention is directed to an isolated nucleic acid sequence comprising from about 10 to about 400 consecutive nucleotides from SEQ ID NO: 1 or a sequence complementary to any one of SEQ ID NO: 1. In one embodiment, the invention is directed to an isolated nucleic acid sequence comprising from about 10 to about 500 consecutive nucleotides from any one of SEQ ID NO: 1 or a sequence complementary to any one of SEQ ID NO: 1. In one embodiment, the invention is directed to an isolated nucleic acid sequence comprising from about 10 to about 600 consecutive nucleotides from any one of SEQ ID NO: 1 or a sequence complementary to any one of SEQ ID NO: 1. In one embodiment, the invention is directed to an isolated nucleic acid sequence comprising from about 10 to about 700 consecutive nucleotides from any one of SEQ ID NO: 1 or a sequence complementary to any one of SEQ ID NO: 1. In one embodiment, the invention is directed to an isolated nucleic acid sequence comprising from about 10 to about 800 consecutive nucleotides from any one of SEQ ID NO: 1 or a sequence complementary to any one of SEQ ID NO: 1. In one embodiment, the invention is directed to an isolated nucleic acid sequence comprising from about 10 to about 900 consecutive nucleotides from any one of SEQ ID NO: 1 or a sequence complementary to any one of SEQ ID NO: 1. In one embodiment, the invention is directed to an isolated nucleic acid sequence comprising from about 10 to about 1000 consecutive nucleotides from any one of SEQ ID NO: 1 or a sequence complementary to any one of SEQ ID NO: 1. In one embodiment, the invention is directed to an isolated nucleic acid sequence comprising from about 10 to about 1500 consecutive nucleotides from any one of SEQ ID NO: 1 or a sequence complementary to any one of SEQ ID NO: 1. In one embodiment, the invention is directed to an isolated nucleic acid sequence comprising from about 10 to about 2000 consecutive nucleotides from any one of SEQ ID NO: 1 or a sequence complementary to any one of SEQ ID NO: 1. In one embodiment, the invention is directed to an isolated nucleic acid sequence comprising from about 10 to about 2500 consecutive nucleotides from any one of SEQ ID NO: 1 or a sequence complementary to any one of SEQ ID NO: 1. In one embodiment, the invention is directed to an isolated nucleic acid sequence comprising from about 10 to about 3000 consecutive nucleotides from any one of SEQ ID NO: 1 or a sequence complementary to any one of SEQ ID NO: 1. In one embodiment, the invention is directed to an isolated nucleic acid sequence comprising from about 10 to about 3500 consecutive nucleotides from any one of SEQ ID NO: 1 or a sequence complementary to any one of SEQ ID NO: 1. In one embodiment, the invention is directed to an isolated nucleic acid sequence comprising from about 10 to about 3600 consecutive nucleotides from any one of SEQ ID NO: 1 or a sequence complementary to any one of SEQ ID NO: 1. In one embodiment, the invention is directed to an isolated nucleic acid sequence comprising from about 10 to about 3621 consecutive nucleotides from any one of SEQ ID NO: 1 or a sequence complementary to any one of SEQ ID NO: 1.

In a further aspect, the invention provides a primer set for determining the presence or absence of the TVHV in a biological sample, wherein the primer set comprises at least one synthetic nucleic acid sequence selected from the group consisting of: a synthetic nucleic acid which has a sequence consisting of from about 10 to about 30 consecutive nucleotides from a nucleic acid sequence of SEQ ID NO: 1, a synthetic nucleic acid which has a sequence consisting of from about 10 to about 30 consecutive nucleotides from a nucleic acids sequence which is complementary to a nucleic acid sequence of SEQ ID NO: 1. In one embodiment, the biological sample is derived from an animal suspected of having the TVHV.

In an further aspect, the invention provides a method for determining the presence or absence of a TVHV in a biological sample, the method comprising: a) contacting nucleic acid from a biological sample with at least one primer which is a nucleic acid sequence consisting of from about 10 to about 30 consecutive nucleotides from a nucleic acids sequence which is complementary to a nucleic acid sequence of SEQ ID NO: 1, b) subjecting the nucleic acid and the primer to amplification conditions, and c) determining the presence or absence of amplification product, wherein the presence of amplification product indicates the presence of RNA associated with TVHV in the sample. In one embodiment, the biological sample is derived from a animal suspected of having a TVHV.

In another aspect, the invention provides a method for determining the presence or absence of the TVHV in a biological sample, the method comprising: a) contacting nucleic acid from a biological sample with at least one primer which is a synthetic nucleic acid which has a sequence consisting of from about 10 to about 30 consecutive nucleotides from a nucleic acid sequence of SEQ ID NO: 1, b) subjecting the nucleic acid and the primer to amplification conditions, and c) determining the presence or absence of amplification product, wherein the presence of amplification product indicates the presence of RNA associated with TVHV in the sample.

In still a further aspect, the invention provides for an interfering RNA (iRNA) comprising a sense strand having at least 15 contiguous nucleotides complementary to a nucleic acid sequence of SEQ ID NO: 1.

In still another aspect, the invention provides a method of reducing the levels of a viral protein, viral mRNA or viral titer in a cell in an animal comprising: administering at least one iRNA agent to an animal, wherein the iRNA agent comprising a sense strand having at least 15 contiguous nucleotides complementary to gene from a TVHV comprising any of SEQ ID NO: 1 and an antisense strand having at least 15 contiguous nucleotides complementary to the sense strand. In one embodiment, the iRNA agent is administered to an animal. In another embodiment, the iRNA agent is administered via nebulization to an animal. In yet another embodiment, the method further comprises co-administering a second iRNA agent to the animal, wherein the second iRNA agent comprising a sense strand having at least 15 or more contiguous nucleotides complementary to second gene from the TVHV, and an antisense strand having at least 15 or more contiguous nucleotides complementary to the sense strand.

In another aspect, the invention provides a method of reducing the levels of a viral protein in a cell in an animal comprising the step of administering an iRNA agent to an animal, wherein the iRNA agent comprises a sense strand having at least 15 or more contiguous nucleotides complementary to a gene from a TVHV comprising SEQ ID NO: 1 and an antisense strand having at least 15 or more contiguous nucleotides complementary to the sense strand.

In certain aspects, the invention is directed to iRNA molecules which target nucleic acids from TVHV, for example but not limited to SEQ ID NO: 1, and variants thereof, and silence a target gene.

An “iRNA agent” (abbreviation for “interfering RNA agent”) as used herein, is an RNA agent, which can down-regulate the expression of a target gene, e.g. a TVHV gene. An iRNA agent may act by one or more of a number of mechanisms, including post-transcriptional cleavage of a target mRNA sometimes referred to in the art as RNAi, or pre-transcriptional or pre-translational mechanisms. An iRNA agent can be a double stranded (ds) iRNA agent.

A “ds iRNA agent” (abbreviation for “double stranded iRNA agent”), as used herein, is an iRNA agent which includes more than one, and in certain embodiments two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g. by a linker, e.g. a polyethyleneglycol linker, to form but one molecule. At least one strand can include a region which is sufficiently complementary to a target RNA. Such strand is termed the “antisense strand”. A second strand comprised in the dsRNA agent which comprises a region complementary to the antisense strand is termed the “sense strand”. However, a ds iRNA agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

iRNA agents as described herein, including ds iRNA agents and siRNA agents, can mediate silencing of a gene, e.g., by RNA degradation. For convenience, such RNA is also referred to herein as the RNA to be silenced. Such a gene is also referred to as a target gene. In certain embodiments, the RNA to be silenced is a gene product of a TVHV gene.

As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secret at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

In the anti viral uses of the present invention, silencing of a target gene can result in a reduction in “viral titer” in the cell or in the animal, wherein “reduction in viral titer” refers to a decrease in the number of viable virus produced by a cell or found in an organism undergoing the silencing of a viral target gene. Reduction in the cellular amount of virus produced can lead to a decrease in the amount of measurable virus produced in the tissues of an animal undergoing treatment and a reduction in the severity of the symptoms of the viral infection. iRNA agents of the present invention are also referred to as “antiviral iRNA agents”.

As used herein, a “TVHV gene” refers to any one of the genes identified in the TVHV genome.

In other aspects, the invention provides methods for reducing viral titer in an animal, by administering to an animal, at least one iRNA which inhibits the expression of a TVHV gene.

In other aspects, the invention provides methods for identifying and/or generating anti-viral drugs. For example, in one aspect the invention provides methods for identifying drugs that bind to and/or inhibit the function of the TVHV-encoded proteins of the invention, or that inhibit the replication or pathogenicity of the TVHV of the invention. Methods of identifying drugs that affect or inhibit a particular drug target, such as high throughput drug screening methods, are well known in the art and can readily be applied to the proteins and viruses of the present invention.

In one aspect, the invention provides an isolated TVHV polypeptide encoded by a TVHV nucleic acid sequence in any of SEQ ID NO: 1, or a fragment thereof.

The TVHV polypeptides and amino acid sequences described herein may be useful for, inter alia, expression of TVHV-encoded proteins or fragments, variants, or derivatives thereof, generation of antibodies against TVHV proteins, generation of primers and probes for detecting TVHV and/or for diagnosing TVHV infection, and screening for drugs effective against TVHV as described herein.

In one embodiment, the TVHV polypeptide fragment can be a polypeptide comprising about 8 consecutive amino acids of a TVHV polypeptide described herein. In another embodiment, the fragment can be a polypeptide comprising about 10 consecutive amino acids of a TVHV polypeptide described herein. In another embodiment, the fragment can be a polypeptide comprising about 14 consecutive amino acids of a TVHV polypeptide described herein. In another embodiment, the fragment can be a polypeptide comprising about 16 consecutive amino acids of a TVHV polypeptide described herein. In another embodiment, the fragment can be a polypeptide comprising about 18 consecutive amino acids of a TVHV polypeptide described herein. In another embodiment, the fragment can be a polypeptide comprising about 20 consecutive amino acids of a TVHV polypeptide described herein. In another embodiment, the fragment can be a polypeptide comprising about 21 or more consecutive amino acids of a TVHV polypeptide described herein.

In yet another embodiment, the TVHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids from a TVHV polypeptide.

In another aspect, the invention provides an isolated TVHV polypeptide encoded by a nucleic acid which comprises consecutive nucleotides having a sequence selected from a TVHV nucleic acid sequence in any of SEQ ID NO: 1, or a fragment thereof.

In another aspect, the invention provides an isolated TVHV polypeptide encoded by a nucleic acid which comprises consecutive nucleotides having a sequence selected from a variant of a TVHV nucleic acid sequence in any of SEQ ID NO: 1 or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In one aspect, the invention provides an isolated TVHV polypeptide encoded by a nucleic acid complementary a TVHV nucleic acid sequence in any of SEQ ID NO: 1, or a fragment thereof.

In another aspect, the invention provides an isolated TVHV polypeptide encoded by a nucleic acid which comprises consecutive nucleotides a TVHV nucleic acid sequence in any of SEQ ID NO: 1, or a fragment thereof.

In another aspect, the invention provides an isolated TVHV polypeptide encoded by a nucleic acid having a sequence substantially identical to a TVHV nucleic acid sequence in any of SEQ ID NO: 1, or a fragment thereof.

In another aspect, the invention provides an isolated TVHV polypeptide encoded by a nucleic acid having a sequence substantially identical to a sequence complementary to a TVHV nucleic acid sequence in any of SEQ ID NO: 1, or a fragment thereof.

In one aspect, the invention provides an isolated TVHV polypeptide having the sequence of any of SEQ ID NOs: 2-17, or a fragment thereof.

In another aspect, the invention provides an isolated TVHV polypeptide which comprises consecutive amino acids having a sequence selected from the group consisting of any of SEQ ID NOs: 2-17, or a fragment thereof.

In another aspect, the invention provides an isolated TVHV polypeptide which comprises consecutive amino acids having a sequence selected from a variant of any of SEQ ID NOs: 2-17, or a fragment thereof. In one embodiment, the variant has at least about 85% identity to SEQ ID NOs: 2-17, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In another aspect, the invention provides an isolated TVHV polypeptide having a sequence substantially identical to a TVHV amino acid sequence in any of SEQ ID NOs: 2-17, or a fragment thereof.

In one aspect, the invention provides an isolated TVHV polypeptide encoded by a TVHV nucleic acid sequence in any of SEQ ID NO: 1, or a fragment thereof.

In one embodiment, the TVHV polypeptide fragment can be a polypeptide comprising about 8 consecutive amino acids of a TVHV polypeptide described herein. In another embodiment, the fragment can be a polypeptide comprising about 10 consecutive amino acids of a TVHV polypeptide described herein. In another embodiment, the fragment can be a polypeptide comprising about 14 consecutive amino acids of a TVHV polypeptide described herein. In another embodiment, the fragment can be a polypeptide comprising about 16 consecutive amino acids of a TVHV polypeptide described herein. In another embodiment, the fragment can be a polypeptide comprising about 18 consecutive amino acids of a TVHV polypeptide described herein. In another embodiment, the fragment can be a polypeptide comprising about 20 consecutive amino acids of a TVHV polypeptide described herein. In another embodiment, the fragment can be a polypeptide comprising about 21 or more consecutive amino acids of a TVHV polypeptide described herein.

In yet another embodiment, the TVHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids from a TVHV polypeptide.

In another aspect, the invention provides an isolated TVHV polypeptide encoded by a nucleic acid which comprises consecutive nucleotides having a sequence selected from a TVHV nucleic acid sequence in any of SEQ ID NO: 1, or a fragment thereof.

In another aspect, the invention provides an isolated TVHV polypeptide encoded by a nucleic acid which comprises consecutive nucleotides having a sequence selected from a variant of a TVHV nucleic acid sequence in any of SEQ ID NO: 1 or a fragment thereof. In one embodiment, the variant has at least about 85% identity to SEQ ID NO: 1, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In one aspect, the invention provides an isolated TVHV polypeptide encoded by a nucleic acid complementary a TVHV nucleic acid sequence in any of SEQ ID NO: 1, or a fragment thereof.

In another aspect, the invention provides an isolated TVHV polypeptide encoded by a nucleic acid which comprises consecutive nucleotides a TVHV nucleic acid sequence in any of SEQ ID NO: 1, or a fragment thereof.

In another aspect, the invention provides an isolated TVHV polypeptide encoded by a nucleic acid having a sequence substantially identical to a TVHV nucleic acid sequence in any of SEQ ID NO: 1, or a fragment thereof.

In another aspect, the invention provides an isolated TVHV polypeptide encoded by a nucleic acid having a sequence substantially identical to a sequence complementary to a TVHV nucleic acid sequence in any of SEQ ID NO: 1, or a fragment thereof.

In one aspect, the invention provides an isolated TVHV polypeptide having the sequence of any of SEQ ID NOs: 2-17, or a fragment thereof.

In another aspect, the invention provides an isolated TVHV polypeptide which comprises consecutive amino acids having a sequence selected from the group consisting of any of SEQ ID NOs: 2-17, or a fragment thereof.

In another aspect, the invention provides an isolated TVHV polypeptide which comprises consecutive amino acids having a sequence selected from a variant of any of SEQ ID NOs: 2-17, or a fragment thereof. In one embodiment, the variant has at least about 85% identity to SEQ ID NOs: 2-17, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1, or a fragment thereof.

In another aspect, the invention provides an isolated TVHV polypeptide having a sequence substantially identical to a TVHV amino acid sequence in any of SEQ ID NOs: 2-17, or a fragment thereof.

The TVHV polypeptides and amino acid sequences described herein may be useful for, inter alia, expression of TVHV-encoded proteins or fragments, variants, or derivatives thereof, and generating vaccines against TVHV.

In one aspect, the invention provides an isolated TVHV polypeptide encoded by a TVHV nucleic acid sequence in any of SEQ ID NO: 1, or a fragment thereof.

In one embodiment, the isolated TVHV polypeptide fragment can be a polypeptide comprising about 8 consecutive amino acids of a TVHV amino acid sequence of any of SEQ ID NOs: 2-17. In another embodiment, the fragment can be a polypeptide comprising about 10 consecutive amino acids of a TVHV amino acid sequence of any of SEQ ID NOs: 2-17. In another embodiment, the fragment can be a polypeptide comprising about 14 consecutive amino acids of a TVHV amino acid sequence of any of SEQ ID NOs: 2-17. In another embodiment, the fragment can be a polypeptide comprising about 16 consecutive amino acids of a TVHV amino acid sequence of any of SEQ ID NOs: 2-17. In another embodiment, the fragment can be a polypeptide comprising about 18 consecutive amino acids of a TVHV amino acid sequence of any of SEQ ID NOs: 2-17. In another embodiment, the fragment can be a polypeptide comprising about 20 consecutive amino acids of a TVHV amino acid sequence of any of SEQ ID NOs: 2-17. In another embodiment, the fragment can be a polypeptide comprising about 21 or more consecutive amino acids of a TVHV amino acid sequence of any of SEQ ID NOs: 2-17.

In yet another embodiment, the isolated TVHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids of a TVHV amino acid sequence of any of SEQ ID NOs: 2-17.

In another aspect, the invention provides an isolated TVHV polypeptide which comprises consecutive amino acids having a sequence selected from a TVHV amino acid sequence of any of SEQ ID NOs: 2-17.

In another aspect, the invention provides an isolated TVHV polypeptide which comprises consecutive nucleotides having a sequence selected from a variant a TVHV amino acid sequence of any of SEQ ID NOs: 2-17, or a fragment thereof. In one embodiment, the variant has at least about 85% identity to any of SEQ ID NOs: 2-17, or a fragment thereof. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to any of SEQ ID NOs: 2-17, or a fragment thereof.

In another aspect, the invention provides an isolated TVHV polypeptide substantially identical to variant a TVHV amino acid sequence of any of SEQ ID NOs: 2-17, or a fragment thereof.

“Substantially identical,” in the context of two nucleic acids or polypeptides, refers to two or more sequences or subsequences that have at least of at least 98%, at least 99% or higher nucleotide or amino acid residue identity, when compared and aligned for maximum correspondence, as measured using one of the following sequence comparison algorithms or by visual inspection. Thus, in certain embodiments, polypeptides that a substantially identical to the TVHV polypeptides described herein can also be used to generate antibodies that bind to the TVHV polypeptides described herein.

“Percent identity” in the context of two or more nucleic acids or polypeptide sequences, refers to the percentage of nucleotides or amino acids that two or more sequences or subsequences contain which are the same. A specified percentage of amino acid residues or nucleotides can have a specified identity over a specified region, when compared and aligned for maximum correspondence over a comparison window, or designated region as measured using one of the following sequence comparison algorithms or by manual alignment and visual inspection. In one aspect, the invention provides a TVHV polypeptide which is a variant of a TVHV polypeptide and has at least about 90%, about 95.5%, about 96%, about 96.5%, about 97%, about 97.5%, about 98%, about 98.5%, about 99%, about 99.5% or about 99.9% identity to a TVHV polypeptide shown in SEQ ID NOs 2-17.

It will be understood that, for the particular TVHV polypeptides described here, natural variations can exist between individual TVHV strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino 15 acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions 20 include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity. It is know that polypeptide sequences having one or more amino acid sequence variations as compared to a reference polypeptide may still be useful for generating antibodies that bind the reference polypeptide. Thus in certain embodiments, the TVHV polypeptides and the antibodies and antibody generation methods related thereto encompass TVHV polypeptides isolated from different virus isolates that have sequence identity levels of at least about 90%, while still representing the same TVHV protein with the same immunological characteristics.

The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

An example of an algorithm that is suitable for determining percent sequence identity and sequence similarity is the FASTA algorithm, which is described in Pearson, W. R. & Lipman, D. J., Proc. Natl. Acad. Sci. U.S.A. 85: 2444, 1988. See also W. R. Pearson, Methods Enzymol. 266: 227-258, 1996. Exemplary parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another example of algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www ncbi.nlm.nih.gov/). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of an algorithm that is suitable for multiple DNA and amino acid sequence alignments is the CLUSTALW program (Thompson, J. D. et al., Nucl. Acids. Res. 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89:10915-10919, 1992).

In yet a further aspect, the invention provides a computer readable medium having stored thereon (i) a nucleic acid sequence selected from the group consisting of: a TVHV nucleic acid sequence in any of SEQ ID NO: 1, a sequence substantially identical to a TVHV nucleic acid sequence in any of SEQ ID NO: 1; a sequence variant of a TVHV nucleic acid sequence in any of SEQ ID NO: 1; or (ii) an amino acid sequence encoded by a nucleic acid sequence selected from the group consisting of: a TVHV nucleic acid sequence in any of SEQ ID NO: 1, an amino acid sequence encoded by a sequence substantially identical to a TVHV nucleic acid sequence in any of SEQ ID NO: 1; an amino acid sequence encoded by a sequence variant of a TVHV nucleic acid sequence in any of SEQ ID NO: 1.

The polypeptides described herein can be used for raising antibodies (e.g. for vaccination purposes). In one aspect, the invention provides antibody that binds a TVHV polypeptide, a TVHV polypeptide fragment or a TVHV polypeptide variant, or a polypeptide substantially identical to a TVHV polypeptide and wherein the antibody is a vaccine antibody that inhibits, neutralizes or reduces the activity or function of the polypeptide or a TVHV. In some embodiments, the antibody is a polyclonal antibody, a monoclonal antibody, an avian antibody or a chimeric antibody.

In still a further aspect, the invention provides a TVHV immunogenic composition comprising a TVHV polypeptide, a TVHV polypeptide fragment or a TVHV polypeptide variant, or a polypeptide substantially identical to a TVHV polypeptide.

As used herein, the term “immunogenic polypeptide” refers to a TVHV polypeptide, or a fragment of a TVHV polypeptide capable of inducing an immune response in a vertebrate host (e.g. a turkey). The term “immunogenic polypeptide” also refers to a TVHV polypeptide, or a fragment of a TVHV polypeptide that can be used to generate an antibody against the TVHV polypeptide, or a fragment of a TVHV polypeptide using other antibody generation techniques known in the art, including, but not limited to, hybridoma, phage display and ribosome display technologies.

In still a further aspect, the invention provides a TVHV vaccine composition comprising a TVHV nucleic acid, a TVHV nucleic acid fragment or a TVHV nucleic acid variant, a nucleic acid substantially identical to a TVHV nucleic acid, a TVHV polypeptide, a TVHV polypeptide fragment or a TVHV polypeptide variant, or a polypeptide substantially identical to a TVHV polypeptide.

One of skill in the art will recognize that when polypeptides are used for raising antibodies, it is not necessary to use the entire polypeptide to generate an antibody capable of recognizing the full length polypeptide. In certain aspects, the invention is directed to methods for generating antibodies that bind to the TVHV polypeptides described herein by generating antibodies that bind to a fragment of a polypeptide described herein. Thus, in one aspect, the invention relates to vaccines for combating TVHV infection, that comprise a protein or immunogenic fragments of a TVHV polypeptide. Still another embodiment of the present invention relates to the TVHV proteins described herein or immunogenic fragments thereof for use in a vaccine. In still another embodiment, the invention relates to the use of the TVHV proteins described herein or immunogenic fragments thereof for the manufacturing of a vaccine for combating TVHV infections.

In one embodiment, the TVHV immunogenic compositions and TVHV vaccines described herein are capable of ameliorating the symptoms of a TVHV infection and/or of reducing the duration of a TVHV infection. In another embodiment, the immunogenic compositions are capable of inducing protective immunity against TVHV infection. The immunogenic compositions of the invention can be effective against the TVHV disclosed herein, and may also be cross-reactive with, and effective against, multiple different clades and strains of TVHV, and against other picornavirus-like viruses.

In other aspect, the invention provides a nucleic acid vector comprising a TVHV nucleic acid sequence, a TVHV nucleic acid fragment or a TVHV nucleic acid variant, or a nucleic acid substantially identical to a TVHV nucleic acid.

In another aspect, the invention provides a nucleic acid vector encoding a TVHV polypeptide, a TVHV polypeptide fragment or a TVHV polypeptide variant, or a polypeptide substantially identical to a TVHV polypeptide. Non-limiting examples of vectors include, but are not limited to retroviral, adenoviral, adeno-associated viral, lentiviral, and vesiculostomatitis viral vectors.

In yet another aspect, the invention provides a host organism comprising a nucleic acid vector encoding a TVHV polypeptide, a TVHV polypeptide fragment or a TVHV polypeptide variant, or a polypeptide substantially identical to a TVHV polypeptide. In one embodiment, the host organism is a prokaryote, a eukaryote, or a fungus. In another embodiment the organism is avian (e.g. a turkey).

In another aspect, the invention provides a method of inducing an immune response in an animal (e.g. a salmon), the method comprising administering a TVHV nucleic acid, a TVHV polypeptide or a TVHV immunogenic composition to the animal. Methods for administering polypeptides to animals (e.g. turkeys), and methods for generating immune responses in animals (e.g. turkeys) by administering immunogenic peptides in immunogenically effective amounts are known in the art.

The polypeptides described herein can be used in the form of a TVHV immunogenic composition to vaccinate an animal (e.g. a turkey) according to any method known in the art. An immunogenic composition for use in vaccination can also include attenuated live viral vaccines, inactivated (killed) viral vaccines, and subunit vaccines. In certain embodiments, TVHVs may be attenuated by removal or disruption of viral sequences whose products cause or contribute to the disease and symptoms associated with TVHV infection, and leaving intact those sequences required for viral replication. In this way an attenuated TVHV can be produced that replicates in animals, and induces an immune response in animals, but which does not induce the deleterious disease and symptoms usually associated with TVHV infection. One of skill in the art can determine which TVHV sequences can or should be removed or disrupted, and which sequences should be left intact, in order to generate an attenuated TVHV suitable for use as a vaccine. TVHV vaccines may also comprise inactivated TVHV, such as by chemical treatment, to “kill” the viruses such that they are no longer capable of replicating or causing disease in animals, but still induce an immune response in an animal (e.g. a salmon). There are many suitable viral inactivation methods known in the art and one of skill in the art can readily select a suitable method and produce an inactivated “killed” TVHV suitable for use as a vaccine.

Methods of purification of polypeptides and of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000. See also, O'Neil et al., “Virus Harvesting and Affinity Based Liquid Chromatography. A Method for Virus Concentration and Purification”, Biotechnology (1993) 11:173-177; Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-211; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fiber (improved BMM hollow fiber) for virus removal” Polymer Journal (1990) 22(12):1085-1100; and Makino et al., “Concentration of live retrovirus with a regenerated cellulose hollow fiber, BMM”, Archives of Virology (1994) 139(1-2):87-96.).

Polypeptides and viruses can be purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Anion exchange resins that may be used include DEAE, EMD TMAE. Cation exchange resins may comprise a sulfonic acid-modified surface. Viruses can be purified using ion exchange chromatography comprising a strong anion exchange resin (e.g. EMD TMAE) for the first step and EMD-SO₃ (cation exchange resin) for the second step. A metal-binding affinity chromatography step can optionally be included for further purification. (See, e.g., WO 97/06243).

A resin such as Fractogel EMD can also be used This synthetic methacrylate based resin has long, linear polymer chains covalently attached and allows for a large amount of sterically accessible ligands for the binding of biomolecules without any steric hindrance.

Column-based liquid affinity chromatography is another purification method that can be used invention. One example of a resin for use in purification method is Matrex Cellufine Sulfate (MCS). MCS consists of a rigid spherical (approx. 45-105 μm diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

Inactivated viruses may be further purified by gradient centrifugation, or density gradient centrifugation. For commercial scale operation a continuous flow sucrose gradient centrifugation would be an option. This method can be used to purify antiviral vaccines and is known to one skilled in the art.

Additional purification methods which may be used to purify viruses of the invention include the use of a nucleic acid degrading agent, a nucleic acid degrading enzyme, such as a nuclease having DNase and RNase activity, or an endonuclease, such as from Serratia marcescens, membrane adsorbers with anionic functional groups or additional chromatographic steps with anionic functional groups (e.g. DEAE or TMAE). An ultrafiltration/dialfiltration and final sterile filtration step could also be added to the purification method.

The purified immunogenic preparations described herein can be substantially free of contaminating proteins derived from the cells or cell culture and can comprise less than about 1000, 500, 250, 150, 100, or 50 pg cellular nucleic acid/μg virus antigen, and less than about 1000, 500, 250, 150, 100, or 50 pg cellular nucleic acid/dose.

In one aspect, vaccination of animals may be performed by directly injecting the TVHV polypeptides, fragments or variants thereof into the animal to generate an immunogenic response. In certain embodiments, the TVHV polypeptides can be injected by themselves, or as immunogenic TVHV compositions comprising other components, including, for example, excipients, additives and adjuvants.

To produce the immunogenic preparations described herein, the TVHV nucleic acid sequences of the invention can be delivered to cultured cells, for example by transfecting cultured cells with plasmids or expression vectors containing TVHV nucleic acid sequences, or by infecting cultured cells with recombinant viruses containing TVHV nucleic acid sequences. TVHV polypeptides may then be expressed in a host cell or expression system and purified. A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, Chinese Hamster Ovary cells (CHO) or Crandell Feline Kidney-cells, also with appropriate vectors or recombinant viruses. In vitro expression systems, such as in-vitro transcription and in-vitro translation systems can also be used to generate the TVHV polypeptides described herein. The purified proteins can then be incorporated into compositions suitable for administration to animals. Methods and techniques for expression and purification of recombinant proteins are well known in the art, and any such suitable methods may be used.

Vaccination may also be performed by direct vaccination with a DNA encoding a TVHV polypeptide. When using such vaccines, the nucleic acid is administered to the animal, and the immunogenic polypeptide(s) encoded by the nucleic acid are expressed in the animal, such that an immune response against the proteins or peptides is generated in the animal. Subunit vaccines may also be proteinaceous vaccines, which contain the viral proteins or subunits themselves, or portions of those proteins or subunits. Any suitable plasmid or expression vector capable of driving expression of a polypeptide may be used. Plasmids and expression vectors can include a promoter for directing transcription of the nucleic acid. The nucleic acid sequence encoding TVHV polypeptides may also be incorporated into a suitable recombinant virus for administration to the animal. Examples of suitable viruses include, but are not limited to, vaccinia viruses, retroviruses, adenoviruses and adeno-associated viruses. One of skill in the art will be able to select a suitable plasmid, expression vector, or recombinant virus for delivery of the TVHV nucleic acid sequences of the invention. Direct vaccination with DNA encoding proteins has been successful for many different proteins. (As reviewed in e.g. Donnelly et al. The Immunologist 2: 20-26 (1993)).

Vaccination with the TVHV nucleic acids and polypeptides described herein can also be performed using live recombinant carriers capable of expressing the polypeptides described herein. Live recombinant carriers are micro-organisms or viruses in which additional genetic information, e.g. a nucleic acid sequence encoding a TVHV polypeptide, or a fragment thereof has been cloned. Animals infected with such live recombinant carriers will produce an immunological response not only against the immunogens of the carrier, but also against the TVHV polypeptide or TVHV polypeptide fragment. Non-limiting examples of live recombinant carriers suitable for use with the methods described herein includes Vibrio anguillarum (Singer, J. T. et al. New Developments in Marine Biotechnology, p. 303-306, Eds. Le Gal and Halvorson, Plenum Press, New York, 1998), and alphavirus-vectors (Sondra Schlesinger and Thomas W. Dubensky Jr. Alphavirus vectors for gene expression and vaccines. Current opinion in Biotechnology, 10:434439 (1999)

Alternatively, passive vaccination can be performed by raising TVHV antibodies in a first animal species (e.g. a rabbit), from antibody-producing cell lines, or from in-vitro techniques before administering such antibodies (in purified or unpurified form) to second animal species (e.g. a turkey). This type of passive vaccination can be used when the second animal is already infected with a TVHV. In some cases, passive vaccination can be useful where the infection in the second animal cannot, or has not had sufficient time to mount an immune response to the infection.

Many methods for the vaccination of avians (e.g. turkeys) are known in the art. For example, Vaccination with the TVHV nucleic acids and polypeptides described herein can be performed in turkeys by injection or through oral administration, however any other method known in the art can also as a method for vaccination. The administration protocol can be optimized in accordance with standard vaccination practice

For oral vaccination, the TVHV nucleic acids, polypeptides or immunogenic compositions described herein can be mixed with feed, coated on the feed or be administered in an encapsulated form. In certain embodiments, vaccination may be performed by incubating live in a TVHV vaccine suspension prior to feeding an animal (e.g. a turkey) such that ingestion of the live feed will cause the TVHV vaccine to accumulate in the digestive tract of the animal undergoing vaccination. One skilled in the art will appreciate that these methods of administration may expose an antigen to potential breakdown or denaturation and thus the skill artisan will ensure that the method of vaccination will be appropriate for a chosen antigen. In the case of oral vaccination, the vaccine may also be mixed with one or more carriers. Carriers suitable for use in oral vaccination include both metabolizable and non-metabolizable substances.

The TVHV nucleic acids, polypeptides or immunogenic compositions described herein can also be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges may be formulated containing a powder mix of the compound and a suitable powder base such as lactose or starch.

The TVHV nucleic acids, polypeptides or immunogenic compositions described herein can be administered in any immunologically effective amount sufficient to trigger an immune response in an animal. In certain instances, this amount can be between about 0.01 and about 1000 micrograms of the TVHV nucleic acid, polypeptide or immunogenic composition per animal.

As used herein, the term “immunologically effective amount” refers to an amount capable of inducing, or enhancing the induction of, the desired immune response in an animal. The desired response may include, inter alia, inducing an antibody or cell-mediated immune response, or both. The desired response may also be induction of an immune response sufficient to ameliorate the symptoms of a TVHV infection, reduce the duration of a TVHV infection, and/or provide protective immunity in an animal against subsequent challenge with a TVHV. An immunologically effective amount may be an amount that induces actual “protection” against TVHV infection, meaning the prevention of any of the symptoms or conditions resulting from TVHV infection in animals. An immunologically effective amount may also be an amount sufficient to delay the onset of symptoms and conditions associated with infection, reduce the degree or rate of infection, reduce in the severity of any disease or symptom resulting from infection, and reduce the viral load of an infected animal.

One of skill in the art can readily determine what is an “immunologically effective amount” of the compositions of the invention without performing any undue experimentation. An effective amount can be determined by conventional means, starting with a low dose of and then increasing the dosage while monitoring the immunological effects. Numerous factors can be taken into consideration when determining an optimal amount to administer, including the size, age, and general condition of the animal, the presence of other drugs in the animal, the virulence of the particular TVHV against which the animal is being vaccinated, and the like. The actual dosage is can be chosen after consideration of the results from various animal studies.

The immunologically effective amount of the immunogenic composition may be administered in a single dose, in divided doses, or using a “prime-boost” regimen. The compositions may be administered by any suitable route, including, but not limited to oral, immersion, parenteral, intradermal, transdermal, subcutaneous, intramuscular, intravenous, intraperitoneal, intranasal, oral, or intraocular routes, or by a combination of routes. The skilled artisan will be able to formulate the vaccine composition according to the route chosen.

In addition to vaccination techniques, antibodies that bind TVHV polypeptides described herein can also be generated by any other method known in the art. Exemplary alternative in-vitro antibody generation technologies, transgenic animal technologies and hybridoma technologies. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Methods for generating avian antibodies can also be used to generate the antibodies described herein. In avians, the egg yolk can be used an antibody source (Altchul et al., Nature Genetics, 1994, 6:119-129). For a review of preimmune diversification and antibody generation in avians, see Reynaud et al., Cell 40, 283-291, 1985 and Thompson et al., Cell 48, 369-378, 1987. In birds, the bursa of Fabricius is the site where B cells undergo gene conversion and are selected for the ability to produce antibodies to antigens. Unlike mammals, the generation of antibody binding specificities occurs before hatching rather than throughout their lives. Another difference between avians and mammals is that the major immunoglobulin is IgY rather than IgG. A small version of IgY lacking a full Fc region (IgY(ΔFc)) is also known to be produced in avians. (Zimmerman, et al, (1971) Biochemistry 10: 482-488).

Any methods for producing antibodies in avians can be used to produce the antibodies described herein. Methods for producing antibodies from avians are known in the art include, but are not limited to those described in WO 94/24268, WO 99/02188 and U.S. patent application Ser. No. 11/095,493.

Antibodies useful in the embodiments of the invention can be derived in several ways well known in the art. In one aspect, the antibodies can be obtained using any of the techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Methods for purifying IgY from egg yolk sacs are also known in the art. See, for example, Polson et al, Immunol Invest. 1985 Aug.; 14(4):323-7; Akita and Nakai, Immunol Methods. 1993 Apr. 2; 160(2):207-14; Akita and Nakai, J Immunol Methods. 1993 Jun. 18; 162(2):155-64 and U.S. Pat. Nos. 4,357,272, 4,550,019, 5,080,895, 5,420,253 and 5,367,054.

In-vitro technologies suitable for generating TVHV binding antibodies include, but are not limited to, ribosome display, yeast display, and bacterial display technologies. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503). In comparison to hybridoma technology, phage and other antibody display methods afford the opportunity to manipulate selection against the antigen target in vitro and without the limitation of the possibility of host effects on the antigen or vice versa.

For example, antibodies that bind TVHV polypeptides may be obtained by selecting from libraries, e.g. a phage library. A phage library can be created by inserting a library of random oligonucleotides or a library of polynucleotides containing sequences of interest, such as from the B-cells of an immunized animal (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-11). The diversity of a phagemid library can be manipulated to increase and/or alter the immunospecificities of the monoclonal antibodies of the library to produce and subsequently identify additional, desirable, avian antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more framework sequences and introducing collections of CDR cassettes derived from antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other antibody generation techniques suitable for generating antibodies against the TVHV polypeptide, or a fragment of a TVHV polypeptide described herein include, the PEPSCAN technique described in Geysen et al (Patent Application WO 84/03564, Patent Application WO 86/06487, U.S. Pat. No. 4,833,092, Proc. Natl. Acad. Sci. 81: 3998-4002 (1984), J. 1 mm. Meth. 102, 259-274 (1987).

Pepsin or papain digestion of whole antibodies that bind TVHV polypeptides can be used to generate antibody fragments that bind TVHV polypeptides. In particular, an Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An (Fab′)₂ fragment of an antibody can be obtained by treating a whole antibody molecule with the enzyme pepsin, without subsequent reduction. An Fab′ fragment of an antibody molecule can be obtained from (Fab′)₂ by reduction with a thiol reducing agent, which yields a molecule consisting of an intact light chain and a portion of a heavy chain. Two Fab′ fragments are obtained per antibody molecule treated in this manner.

Antibodies can be produced through chemical crosslinking of the selected molecules (which have been produced by synthetic means or by expression of nucleic acid that encode the polypeptides) or through recombinant DNA technology combined with in vitro, or cellular expression of the polypeptide, and subsequent oligomerization. Antibodies can be similarly produced through recombinant technology and expression, fusion of hybridomas that produce antibodies with different epitope specificities, or expression of multiple nucleic acid encoding antibody variable chains with different epitopic specificities in a single cell.

Antibodies may be either joined directly or indirectly through covalent or non-covalent binding, e.g. via a multimerization domain, to produce multimers. A “multimerization domain” mediates non-covalent protein-protein interactions. Specific examples include coiled-coil (e.g., leucine zipper structures) and alpha-helical protein sequences. Sequences that mediate protein-protein binding via Van der Waals' forces, hydrogen bonding or charge-charge bonds can also be used as multimerization domains. Additional examples include basic-helix-loop-helix domains and other protein sequences that mediate heteromeric or homomeric protein-protein interactions among nucleic acid binding proteins (e.g., DNA binding transcription factors, such as TAFs). One specific example of a multimerization domain is p53 residues 319 to 360 which mediate tetramer formation. Another example is human platelet factor 4, which self-assembles into tetramers. Yet another example is extracellular protein TSP4, a member of the thrombospondin family, which can form pentamers. Additional specific examples are the leucine zippers of jun, fos, and yeast protein GCN4.

Antibodies may be directly linked to each other via a chemical cross linking agent or can be connected via a linker sequence (e.g., a peptide sequence) to form multimers.

The antibodies described herein can be polyclonal or monoclonal. The antibodies can also be chimeric (i.e., a combination of sequences from more than one species, for example, a chimeric mouse-salmon immunoglobulin). Species specific antibodies avoid certain of the problems associated with antibodies that possess variable and/or constant regions from other species. The presence of such protein sequences from other species can lead to the rapid clearance of the antibodies or can lead to the generation of an immune response against the antibody by an antibody.

The antibodies described herein can be antibodies that bind to other molecules (antigens) via heavy and light chain variable domains, V_{H} and V_{L}, respectively. The antibodies described herein include, but are not limited to IgY, IgY(ΔFc)), IgG, IgD, IgA, IgM, IgE, and IgL. The antibodies may be intact immunoglobulin molecules, two full length heavy chains linked by disulfide bonds to two full length light chains, as well as subsequences (i.e. fragments) of immunoglobulin molecules, with or without constant region, that bind to an epitope of an antigen, or subsequences thereof (i.e. fragments) of immunoglobulin molecules, with or without constant region, that bind to an epitope of an antigen. Antibodies may comprise full length heavy and light chain variable domains, V_{H} and V_{L}, individually or in any combination.

The basic immunoglobulin (antibody) structural unit can comprise a tetramer. Each tetramer can be composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (Vₗ) and variable heavy chain (V_{H}) refer to these light and heavy chains respectively.

The antibodies described herein may exist as intact immunoglobulins or as a number of well characterized fragments produced by digestion with various peptidases. In particular, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology. The Fab′ regions may be derived from antibodies of animal or human origin or may be chimeric (Morrison et al., Proc Natl. Acad. Sci. USA 81, 6851-11855 (1984) both incorporated by reference herein) (Jones et al., Nature 321, 522-525 (1986), and published UK patent application No. 8707252, both incorporated by reference herein).

The antibodies described herein can include or be derived from any mammal, such as but not limited to, an avian, a human, a mouse, a rabbit, a rat, a rodent, a primate, or any combination thereof and includes isolated avian, human, primate, rodent, mammalian, chimeric, humanized and/or CDR-grafted or CDR-adapted antibodies, immunoglobulins, cleavage products and other portions and variants thereof. In one embodiment the antibody is purified.

The antibodies described herein include full length antibodies, subsequences (e.g., single chain forms), dimers, trimers, tetramers, pentamers, hexamers or any other higher order oligomer that retains at least a part of antigen binding activity of monomer. Multimers can comprise heteromeric or homomeric combinations of full length antibody, subsequences, unmodified or modified as set forth herein and known in the art. Antibody multimers are useful for increasing antigen avidity in comparison to monomer due to the multimer having multiple antigen binding sites. Antibody multimers are also useful for producing oligomeric (e.g., dimer, trimer, tertamer, etc.) combinations of different antibodies thereby producing compositions of antibodies that are multifunctional (e.g., bifunctional, trifunctional, tetrafunctional, etc.).

Specific examples of antibody subsequences include, for example, Fab, Fab′, (Fab′)₂, Fv, or single chain antibody (SCA) fragment (e.g., scFv). Subsequences include portions which retain at least part of the function or activity of full length sequence. For example, an antibody subsequence will retain the ability to selectively bind to an antigen even though the binding affinity of the subsequence may be greater or less than the binding affinity of the full length antibody.

An Fv fragment is a fragment containing the variable region of a light chain V_{L} and the variable region of a heavy chain V_{H} expressed as two chains. The association may be non-covalent or may be covalent, such as a chemical cross-linking agent or an intermolecular disulfide bond (Inbar et al., (1972) Proc. Natl. Acad. Sci. USA 69:2659; Sandhu (1992) Crit. Rev. Biotech. 12:437).

Other methods of producing antibody subsequences, such as separation of heavy chains to form monovalent light-heavy chain fragments, further cleavage of fragments, or other enzymatic, chemical, or genetic techniques may also be used, provided that the subsequences bind to the antigen to which the intact antibody binds.

A single chain antibody (“SCA”) is a genetically engineered or enzymatically digested antibody containing the variable region of a light chain V_{L} and the variable region of a heavy chain, optionally linked by a flexible linker, such as a polypeptide sequence, in either V_{L}-linker-V_{H} orientation or in V_{H}-linker-V_{L} orientation. Alternatively, a single chain Fv fragment can be produced by linking two variable domains via a disulfide linkage between two cysteine residues. Methods for producing scFv antibodies are described, for example, by Whitlow et al., (1991) In: Methods: A Companion to Methods in Enzymology 2:97; U.S. Pat. No. 4,946,778; and Pack et al., (1993) Bio/Technology 11:1271.

The TVHV nucleic acids, polypeptides and immunogenic compositions described herein can be used to generate antibodies that that inhibit, neutralize or reduce the activity or function of a polypeptide or a TVHV. In certain aspects, the invention is directed to a method for treating an animal (e.g. a salmon), the method comprising administering to the animal TVHV nucleic acids, polypeptides and immunogenic compositions, or administering to the animal an antibody which specifically binds to a TVHV polypeptide such that the activity or function of a TVHV polypeptide or a TVHV is inhibited, neutralized or reduced.

In another aspect, the invention described herein relates to TVHV immunogenic compositions comprising TVHV polypeptides or TVHV nucleic acids. In some embodiments, the TVHV immunogenic compositions can further comprise carriers, adjuvants, excipients and the like. The TVHV immunogenic compositions described herein can be formulated readily by combining the active compounds with immunogenically acceptable carriers well known in the art. The TVHV immunogenic compositions described herein can be formulated in a conventional manner using one or more physiologically acceptable carriers comprising excipients and auxiliaries which facilitate processing of the active compounds into preparations which can be used to induce an immunogenic response. Such carriers can be used to formulate suitable tablets, pills, dragees, capsules, liquids, gels, syrups, slurries, suspensions and the like. In one embodiment, the immunogenic composition can be obtained by solid excipient, grinding a resulting mixture, and processing the mixture of granules, after adding suitable auxiliaries, if desired, to obtain tablets or dragee cores.

The immunogenic composition described herein can be manufactured in a manner that is itself known, e.g. by means of conventional mixing, dissolving, granulating, dragee-making, levigating, emulsifying, encapsulating, entrapping or lyophilizing processes. Proper formulation is dependent upon the route of administration chosen.

When a immunogenetically effective amount of a TVHV immunogenic composition is administered to an animal, the composition can be in the form of a pyrogen-free, parenterally acceptable aqueous solution. The preparation of such parenterally acceptable protein or other active ingredient solutions, having due regard to pH, isotonicity, stability, and the like, is within the skill in the art. For example, TVHV immunogenic compositions described herein can contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The immunogenic composition of the present invention may also contain stabilizers, preservatives, buffers, antioxidants, or other additives known to those of skill in the art. The TVHV immunogenic compositions can be formulated in aqueous solutions, physiologically compatible buffers such as Hanks's solution, Ringer's solution, or physiological saline buffer. For transmucosal administration, penetrants appropriate to the barrier to be permeated are used in the formulation. Such penetrants are generally known in the art.

When the TVHV immunogenic compositions is administered orally, protein or other active ingredient of the present invention can be in the form of a tablet, capsule, powder, solution or elixir. When administered in tablet form, the immunogenic composition of the invention may additionally contain a solid carrier such as a gelatin or an adjuvant.

The TVHV immunogenic compositions described herein can encode or contain any of the TVHV proteins or peptides described herein, or any portions, fragments, derivatives or mutants thereof, that are immunogenic in an animal. One of skill in the art can readily test the immunogenicity of the TVHV proteins and peptides described herein, and can select suitable proteins or peptides to use in subunit vaccines.

The TVHV immunogenic compositions described herein comprise at least one TVHV amino acid or polypeptide, such as those described herein. The compositions may also comprise one or more additives including, but not limited to, one or more pharmaceutically acceptable carriers, buffers, stabilizers, diluents, preservatives, solubilizers, liposomes or immunomodulatory agents. Suitable immunomodulatory agents include, but are not limited to, adjuvants, cytokines, polynucleotide encoding cytokines, and agents that facilitate cellular uptake of the TVHV-derived immunogenic component.

The TVHV immunogenic compositions described herein can also contain an immunostimulatory substance, a so-called adjuvant Adjuvants in general comprise substances that boost the immune response of the host in a non-specific manner. A number of different adjuvants are known in the art.

The TVHV immunogenic compositions described herein may also comprise a so-called “vehicle”. A vehicle is a compound to which the protein adheres, without being covalently bound to it. Such vehicles are e.g. biomicrocapsules, micro-alginates, liposomes and macrosols, all known in the art. A special form of such a vehicle, in which the antigen is partially embedded in the vehicle, is the so-called ISCOM (EP 109.942, EP 180.564, EP 242.380). In addition, the vaccine may comprise one or more suitable surface-active compounds or emulsifiers, e.g. Span or Tween. Certain organic solvents such as dimethylsulfoxide also may be employed.

The TVHV immunogenic compositions described herein can also be mixed with stabilizers, e.g. to protect degradation-prone proteins from being degraded, to enhance the shelf-life of the vaccine, or to improve freeze-drying efficiency. Useful stabilizers are i.e. SPGA (Bovarnik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

When administered in liquid form, a liquid carrier such as water, petroleum, oils of animal or plant origin such as peanut oil, mineral oil, soybean oil, or sesame oil, or synthetic oils may be added. The liquid form of the immunogenic composition may further contain physiological saline solution, dextrose or other saccharide solution, or glycols such as ethylene glycol, propylene glycol or polyethylene glycol. When administered in liquid form, the immunogenic composition contains from about 0.5 to 90% by weight of protein or other active ingredient of the present invention, and from about 1 to 50% protein or other active ingredient of the present invention.

The TVHV immunogenic compositions described herein include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The TVHV immunogenic compositions described herein can also be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

The TVHV immunogenic compositions described herein can also be in the form of a complex of the protein(s) or other active ingredient of present invention along with protein or peptide antigens.

The TVHV immunogenic compositions described herein can be made suitable for parenteral administration and can include aqueous solutions comprising TVHV nucleic acids or polypeptides in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient maybe in powder form for constitution with a suitable vehicle, e.g., sterile pyrogen-free water, before use.

The TVHV immunogenic compositions described herein can also be in the form of a liposome in which protein of the present invention is combined, in addition to other acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The TVHV immunogenic compositions described herein can also be formulated as long acting formulations administered by implantation (for example subcutaneously or intramuscularly) or by intramuscular injection. The compositions may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Carriers for use with the TVHV immunogenic compositions described herein can be a co-solvent systems comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. The proportions of a co-solvent system may be varied considerably without destroying its solubility and toxicity characteristics. The identity of the co-solvent components can also be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

The immunogenic compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with immunogenically compatible counter ions. Such immunogenically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

Excipients suitable for use in the immunogenic compositions described herein include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In certain embodiments, the polypeptides of the present invention can be suitable for use as antigens to detect antibodies against TVHV represented by SEQ ID NO: 1, and variants thereof. In other embodiments, the polypeptides of the present invention which comprise antigenic determinants can be used in various immunoassays to identify animals exposed to and/or samples which comprise TVHV represented by SEQ ID NO: 1, and variants thereof.

In another aspect, the invention provides a method for determining whether or not a sample contains a TVHV, the method comprising: (a) providing an immunoassay comprising an antibody against a TVHV derived antigen, (b) contacting the antibody with a biological sample, (c) detecting binding between antigens in the test sample and the antibody. In one embodiment, the immunoassay is a lateral flow assay or ELISA. In one embodiment, the biological sample is derived from an animal suspected of having a TVHV.

In still a further aspect, the invention provides a method for determining whether or not a sample contains antibodies against TVHV, the method comprising: (a) providing an immunoassay comprising an antigen from a TVHV, (b) contacting the antigen with a biological sample, (c) detecting binding between antibodies in the test sample and the antigen.

The antibodies of the invention can also be used to purify polypeptides of any polypeptide encoded by the nucleic sequence acid of any one of SEQ ID NO: 1, polypeptides comprising the sequence of any of SEQ ID NOs: 2-17, or variants or fragments thereof.

In other embodiments, the antibodies of the invention can be used to identify expression and localization of a TVHV polypeptide or variants or fragments thereof. Analysis of expression and localization of TVHV polypeptides, or variants or fragments thereof, can be useful in diagnosing a TVHV infection or for determining potential role of a TVHV polypeptide.

In other embodiments, the antibodies of the present invention can be used in various immunoassays to identify animals exposed to and/or samples which comprise antigens from TVHV.

Any suitable immunoassay which can lead to formation of antigen-antibody complex can also be used. Variations and different formats of immunoassays, for example but not limited to ELISA, lateral flow assays for detection of analytes in samples, immunoprecipitation, are known in the art. In various embodiments, the antigen and/or the antibody can be labeled by any suitable label or method known in the art. For example enzymatic immunoassays may use solid supports, or immunoprecipitation. Immunoassays which amplify the signal from the antigen-antibody immune complex can also be used with the methods described herein.

In certain aspects the invention provides methods for assaying a sample to determine the presence or absence of a TVHV polypeptide, or a fragment or a variant thereof. In certain embodiments, methods for assaying a sample, include, but are not limited to, methods which can detect the presence of nucleic acids, methods which can detect the presence of TVHV polypeptides, methods which can detect the presence of antibodies against TVHV polypeptides, or any polypeptide encoded by a TVHV nucleic acid.

In still a further aspect, the invention provides a TVHV diagnostic kit comprising a TVHV nucleic acid, a TVHV nucleic acid fragment or a TVHV nucleic acid variant, a nucleic acid substantially identical to a TVHV nucleic acid, or a TVHV antibody.

One of skill in the art will recognize that when antibodies or nucleic acid are used for diagnostic purposes, it is not necessary to use the entire nucleic acid or antibody to detect a TVHV or a TVHV polypeptide in an animal or in a sample. In certain aspects, the invention is directed to methods for generating antibodies that bind to the TVHV polypeptides described herein by generating antibodies that bind to a fragment of a polypeptide described herein. Thus, in one aspect, the invention relates to diagnostic kits for detecting TVHV infection or the presence of TVHV in a sample, that comprise a TVHV nucleic acid or a TVHV antibody.

Other additives that are useful in vaccine formulations are known and will be apparent to those of skill in the art.

The following examples illustrate the present invention, and are set forth to aid in the understanding of the invention, and should not be construed to limit in any way the scope of the invention as defined in the claims which follow thereafter.

### EXAMPLES

### Example 1

### Identification of a Novel Picornavirus in 1 Turkey Poults with Hepatitis

Turkey poult samples were collected between February 2008 and January 2010 from eight different commercial flocks located in California, USA. The flock sizes ranged from 22,500 birds to 40,000 birds. Clinical signs in poults included anorexia, lethargy, diarrhea and increased mortality. Post mortem analyses revealed livers with white foci and occasionally pale patchy areas in the pancreas. Histopathology revealed necrosis of hepatocytes and acinar cells of the pancreas and inflammation.

High throughput pyrosequencing of RNA from livers of turkey poults with turkey viral hepatitis (TVH) showed the presence of picornaviral sequences. Subsequent cloning of the approximately 9 kilobase genomic sequence showed an organization similar to picornaviruses with conservation of motifs within the P1, P2 and P3 genome regions, but also unique features, including a 1.2 kilobase sequence of unknown function at the junction of P1 and P2 regions. Real-time PCR assays confirmed that viral RNA was present in liver, bile, intestine, serum and cloacal swabs of poults with disease.

Analysis of liver sections by in situ hybridization with viral probes and immunohistochemistry with sera from diseased poults demonstrated viral nucleic acid and protein in livers of diseased poults, but not in livers of normal poults. In concert, molecular, anatomic and immunological evidence indicate that TVH is due to infection with a novel picornavirus, tentatively named turkey viral hepatitis-associated virus (TVHV).

### Example 2

### High-Throughput Pyrosequencing

Total RNA was extracted from four 25-day-old turkey poults with disease using TRI Reagent and treated with DNaseI. 2 μg of DNaseI-treated RNA was reverse transcribed using Superscript II kit and random octamer primers with an arbitrary specific anchor sequence as described previously (Palacios et al., Emerg Infect Dis. 2007; 13:73-81). cDNA was RNase H-treated prior to random amplification by PCR. The resulting products were purified using MinElute and ligated to linkers for sequencing on a GSL FLX Sequencer. Sequences were clustered and assembled into contiguous fragments (contigs) following trimming of primer sequences, and Basic Local Alignment Search Tool (BLAST) analysis was applied to compare contigs (or single reads) at nucleotide (nt) and amino acid (aa) level to the GenBank database (http://www ncbi.nlm.nih.gov).

### Example 3

### PCR and Genome Sequencing

Primers were designed that bridged contigs identified by high throughput sequencing. The PCR assays were conducted using HotStar polymerase, primer at 0.2 μM each and 1 μl of random hexamer-primed cDNA. The draft genome sequence was confirmed by selecting additional primers to generate approximately 1 kilobase (kb) products across the entire sequence for direct dideoxy sequencing (Genewiz, South Plainfield, N.J., USA), applying TaKaRa LA Taq polymerase with GC buffer to obtain products in areas that proved difficult to amplify due to potential secondary structures or elevated GC content.

### Example 4

### Quantitative TaqMan Real-Time PCR

Primers and probes for quantitative real-rime PCR were selected within the 5′ untranslated region (UTR) of the turkey viral hepatitis-associated virus (TVHV) genome using Primer Express 1.0 software (Applied Biosystems, Foster City, Calif.). The primer/probe set TVHVforward1 5′-CACCCTCTAYGGGCAATGT-3′, TVHVreverse1 5′-TCAGCCAGTCTATGGCCAGG-3′, and TVHVprobe1 6FAM-5′-TGGATTCCCATCTCACGCGTCCAC-3′-TMR used in Assay 1 (Table 1) was chosen based on initial TVHV strains sequenced. Primer TVHVforward2 5′-CACCCTYYAYGGGCAAATGT-3′ and probe TVHVprobe2 6FAM-5′-ATTCCCATCTCACGCGTCCAC-3′-TMR were later selected to cover for sequence variation of additional strains and used with TVHVreverse1 primer in Assay 2 (Table 2).

Table 1. Real-Time PCR Measurement of Viral Sequences in Turkeys with TVH

TABLE 1
Real-time PCR measurement of viral
sequences in turkeys with TVH

| | | | | | Virus |
|---|---|---|---|---|---|
| | | | | | copies† |
| | | Hepatitis/ | | | (300 ng of |
| Case no. | Age | Control | Organ | Ct* | total RNA) |
| 2993A | 25 day-old | Hepatitis | Liver | 17.12 | 5.66 × 10⁷ |
| 2993B | 25 day-old | Hepatitis | Liver | 17.02 | 6.22 × 10⁷ |
| 2993C | 25 day-old | Hepatitis | Liver | 26.98 | 9.0 × 10³ |
| 2993D | 25 day-old | Hepatitis | Liver | 17.93 | 2.8 × 10⁷ |
| 0091.1 | 28 day-old | Hepatitis | Liver | 17.24 | 5.1 × 10 |
| 0091.2 | 28 day-old | Hepatitis | Liver | 17.69 | 3.43 × 10⁷ |
| 0091.3 | 28 day-old | Hepatitis | Liver | 23.26 | 2.57 × 10⁵ |
| 1813.1 | 26 day-old | Hepatitis | Liver | 23.48 | 2.12 × 10⁵ |
| 1813.2 | 26 day-old | Hepatitis | Liver | 21.23 | 1.53 × 10⁶ |
| 1813.3 | 26 day-old | Hepatitis | Intestine | 20.94 | 1.97 × 10⁶ |
| 0690 | 30 day-old | Hepatitis | Liver | 34.92 | 8.98 × 10⁰ |
| 1999 | 29 day-old | Hepatitis | Liver | 28.44 | 8.42 × 10³ |
| | | | Pancreas | 25.97 | 7.32 × 10⁴ |
| | | | Intestine | 24.85 | 1.96 × 10⁵ |
| 1621.1 | 42 day-old | Non-TVH | Liver | >36§ | Negative |
| 1621.2 | 42 day-old | Non-TVH | Liver | >36§ | Negative |
| 1621.3 | 42 day-old | Non-TVH | Liver | >36§ | Negative |

*Ct, cycle threshold.
†Copy numbers were calculated on the basis of a standard curve generated from cloned target sequences.
§A Ct of >36 was rated as negative based on the highest dilution of standard representing 5 copies

A calibration standard for both assays was generated from strain 2993A by cloning a 571 nt genomic fragment into the pGEM-T Easy vector. PCR assays were pursued in triplicate using a StepOnePlus Real-time PCR system, a standard cycling profile of 45 cycles in a volume of 25 μl containing random hexamer-primed cDNA, 300 nM primer (each) and 200 nM probe. Results were expressed as mean copy number per 300 ng total RNA.

TABLE 2
Real-time PCR measurement of viral sequences in cloacal
swabs, sera and bile from turkeys with TVH.

| | | | | | Virus |
|---|---|---|---|---|---|
| | | | | | copies† |
| | | Hepatitis/ | | | (300 ng of |
| Case no. | Age | Control | Sample | Ct* | total RNA) |
| 2641.1 | 29 day-old | Hepatitis | Cloacal swab | 21.68 | 2.28 × 10⁷ |
| | | | Bile | 31.44 | 7.58 × 10⁴ |
| | | | Sera | 43.12 | 1.2 × 10² |
| 2641.2 | 29 day-old | Hepatitis | Cloacal swab | >44§ | Negative |
| | | | Bile | 32.5 | 4.04 × 10⁴ |
| | | | Sera | 38.04 | 1.67 × 10³ |
| 2641.3 | 29 day-old | Hepatitis | Cloacal swab | >44§ | Negative |
| | | | Bile | 28.60 | 3.84 × 10⁵ |
| | | | Sera | 34.18 | 1.55 × 10⁴ |
| 2641.4 | 29 day-old | Hepatitis | Cloacal swab | 30.6 | 1.21 × 10⁵ |
| | | | Bile | 27.72 | 6.48 × 10⁵ |
| | | | Sera | 41.06 | 3.06 × 10² |
| 2641.5 | 29 day-old | Hepatitis | Cloacal swab | 28.11 | 5.09 × 10⁵ |
| | | | Bile | 32.8 | 5.84 × 10⁴ |
| | | | Sera | 41.33 | 2.71 × 10² |
| 394.1 | 28-day-old | Hepatitis | Cloacal swab | >44§ | Negative |
| | | | Sera | 43.27 | 8.17 × 10¹ |
| 394.2 | 28-day-old | Hepatitis | Cloacal swab | 40.7 | 4.55 × 10² |
| | | | Sera | >44§ | Negative |
| 394.3. | 28-day-old | Hepatitis | Cloacal swab | >44§ | Negative |
| | | | Sera | >44§ | Negative |
| 394.4 | 28-day-old | Hepatitis | Cloacal swab | 29.62 | 2.14 × 10⁵ |
| | | | Sera | >44§ | Negative |
| 394.5 | 28-day-old | Hepatitis | Sera | >44§ | Negative |
| 394.6 | 28-day-old | Hepatitis | Sera | >44§ | Negative |
| 394.7 | 28-day-old | Hepatitis | Sera | >44§ | Negative |
| 394.8 | 28-day-old | Hepatitis | Sera | >44§ | Negative |
| 394.9 | 28-day-old | Hepatitis | Sera | 31.07 | 9.34 × 10⁴ |
| 3302.1 | 39-day-old | Hepatitis | Sera | 36.38 | 3.08 × 10³ |
| 3302.2 | 39-day-old | Hepatitis | Sera | >44§ | Negative |
| 3302.3 | 39-day-old | Hepatitis | Sera | >44§ | Negative |
| 3302.5 | 39-day-old | Hepatitis | Sera | >44§ | Negative |
| 2491.1 | 32-day-old | Non-TVH | Cloacal swab | >44§ | Negative |
| 2491.2 | 32-day-old | Non-TVH | Cloacal swab | 25.67 | 2.2 × 10⁶ |
| 2491.3 | 32-day-old | Non-TVH | Cloacal swab | >44§ | Negative |
| 2491.4 | 32-day-old | Non-TVH | Cloacal swab | >44§ | Negative |
| 2491.5 | 32-day-old | Non-TVH | Cloacal swab | >44§ | Negative |
| 2491.6 | 32-day-old | Non-TVH | Cloacal swab | 34.74 | 1.11 × 10⁴ |
| 407.1 | 39-day-old | Non-TVH | Cloacal swab | >44§ | Negative |
| 407.2 | 39-day-old | Non-TVH | Cloacal swab | >44§ | Negative |
| 407.3 | 39-day-old | Non-TVH | Cloacal swab | >44§ | Negative |
| 407.4 | 39-day-old | Non-TVH | Cloacal swab | >44§ | Negative |

*Ct, cycle threshold.
†Copy numbers were calculated on the basis of a standard curve generated from cloned target sequences.
§A Ct of >44 was rated as negative based on the highest dilution of standard representing 5 copies

### Example 5

### Phylogenetic Analysis

Phylogenetic analyses were performed based on TVHV P1, 2C/3C/3D, and full polyprotein sequence excluding divergent aa 799-1199. Sequences were aligned to selected members of the Picornaviridae family by ClustalW software. Trees were constructed using the Molecular Evolutionary Genetics Analysis (MEGA) software version 4.0.2 (Tamura et al., Molecular Biology and Evolution. 2007; 24:1596-9). A Jukes-Cantor model was applied to calculate distance and statistical significance was assessed by bootstrap re-sampling of 1,000 pseudoreplicate data sets.

### Example 6

### In Situ Hybridization

Viral probes and β-actin control probes were designed and applied according to the QuantiGene ViewRNA protocol using branched DNA technology. 20 viral probes, 17-28 nt in length, were selected that cover 500 nt of target sequence in 2B/2C. 40 β-actin probes, 17-26 nt in length, were selected covering 873 nt of mRNA sequence. 5 μm thick paraffin-embedded tissue sections were fixed, permeabilized with protease and hybridized with oligonucleotides conjugated to alkaline phosphatase. After incubation with FastRed substrate the slides were counterstained with hematoxylin and cover-slipped with Permount. Images were acquired using a Zeiss AX10 Scope AI, ProgRes digital microscope camera and Mac Capture Pro 2.6.0 software.

### Example 7

### Immunohistochemistry

Glass slides with embedded tissues were heated at 56° C. for 10 min and washed in citrus clearing agent to remove paraffin. The sections were rehydrated through graded alcohol solutions. Endogenous peroxidase activity was blocked by incubation in 0.3% H2O2 diluted in methanol for 30 min. Non-specific binding was blocked by incubating sections in 10% goat serum in PBS for 1 h at 37° C. Sera from poults with or without disease were added to the sections at 1:1000 dilution in PBS for overnight incubation at 4° C. Following washes in PBS, horseradish peroxidase (HRP)-labeled goat anti-turkey IgG was added at 1:250 dilution in PBS for 1 h at 37° C. After PBS washes, the Vectastain Elite ABC kit was used, employing 3,\acute{3}-diaminobenzidine tetrahydrochloride as substrate. The sections were counterstained with hematoxylin and dehydrated through graded alcohols. Finally the slides were cover-slipped using Permount and visualized with a Zeiss AX10 Scope AI light microscope at ×40 magnification.

### Example 8

### Identification of TVHV

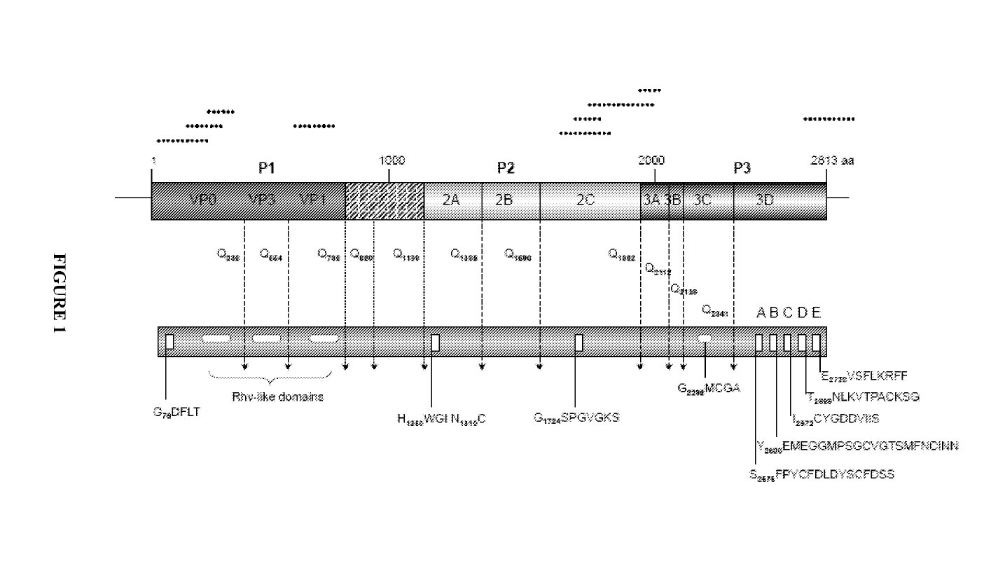

Unbiased high throughput pyrosequencing of RNA extracted from livers of four poults with hepatitis (animals 2993A, 2993B, 2993C and 2993D; Table 1) yielded approximately 63,100 sequence reads with a mean length of 285 nt. Seven contigs (average length of 674 nt, comprising 105 sequence reads) and 2 singletons (read lengths of 486 nt and 498 nt) that together yielded 3,182 nt of sequence, were identified after primer trimming and assembly. Analysis at the nt level was not informative; however, BLASTx analysis revealed significant similarity to picornaviral sequences at the aa level. The remainder of the genome was determined from RNA of poult 2993D by RT-PCR using primers linking the individual contigs and singletons (FIG. 1). Applying additional primers in various genome regions, a 153 second 9 kb genomic sequence was generated from another diseased poult (animal 0091.1). Sequence analysis revealed that the second strain had the same genome organization as strain 2993D and 96.8% aa and 89.9% nt sequence identity (GenBank accessions numbers HM751199 and HQ189775).

### Example 9

### TVHV Genome Organization

The TVHV genome, comprising >9,040 nt and 2813 aa, is larger than that of equine rhinitis virus B (genus Erbovirus), the largest known picornaviral genome (Wutz et al., J. Gen Virol. 1996; 77:1719-30). This chiefly reflects the presence of a 1.2 kb sequence at the junction of the P1 and P2 regions in an otherwise typical picornaviral genome (FIG. 1). The incomplete 461 nt 5′UTR includes a 30 nt motif (nt 270-300 of TVHV) that is identical to duck hepatitis A virus 5′UTR (Avihepatovirus genus), which has a type IV internal ribosome entry site (IRES). However, Mfold-modeling of the available sequence did not allow identification of the IRES type present in TVHV. At the 3′ end, 140 nt of UTR were recovered (without reaching a poly-A tail) that showed no sequence similarity to other picornaviral 3′UTRs.

With the exception of a canonical Gxxx[T/S] myristylation sequence there is little sequence conservation in the VP0 of TVHV with respect to other picornaviruses. Cleavage at Q76/A could generate the N-terminal myristylation site present in many picornaviruses; however, cleavage at this site is not supported by NetPicoRNA prediction and there is no classical leader sequence (FIG. 1; http://www cbs.dtu.dk/services/Net PicoRNA). NetPicoRNA analysis did not indicate a VP2/4 maturation cleavage, as found in the avihepato-, kobu-, and parechoviruses. Sequence comparisons of the P1 region, mainly driven by recognizable sequence conservation in VP3 and a few regions in VP1, indicates highest aa identity (20%) with turdiviruses, unclassified pi 176 cornaviruses recently identified from wild birds (Woo et al., J Gen Virol. 2010: Jun 16). Homology in P1 to pfam sequence cluster cd00205 ‘picornavirus capsid protein domain like’ (accession no. PF00073) was observed between aa residues 109-267, 391-539, and 622-768 (FIG. 1). Potential cleavage sites within P1 are predicted after Q388 (VP0/3) and Q554 (VP3/1).

Protein 2A motifs in TVHV are conserved with respect to kobuviruses after a predicted cleavage site Q1199. However, the sequence lacks the trypsin-like protease motifs that allow autocatalytic cleavage at the N-termini of entero- and sapeloviruses, as well as the NPGP motif that facilitates C-terminal cleavage inaphtho-, avihepato-, cardio-, erbo-, seneca-, and teschoviruses. The predicted 2A sequence of TVHV resembles Hbox-NC motifs present in hepato-, kobu-, parecho-, and tremoviruses (H1250WGI, N1310C followed by a hydrophobic region L1332-V1350). The TVHV 2A protein may be generated by cleavage at conserved protease sites; however, multiple cleavage sites between P1 and P2 are predicted by NetPicoRNA (FIG. 1). Cleavage after Q798 and Q1199 can occur, as it would generate VP1 and 2A products that align with other picornaviral proteins. As a result, one or two additional proteins (depending on cleavage at Q880, FIG. 1) can be produced from this genome region that have no homology to any viral product recorded in GenBank. Multiple 2A1, 2A2, or 2A3 protein products with undefined function are described in Ljungan virus, seal picornavirus, and duck hepatitis virus genomes (Kapoor et al., J. Virol. 2008; 82:311-20; Johansson et al., J Gen Virol. 2003; 84:837-44; Tseng et al., Virus Res. 2007; 126:19-31). Alignment analyses also indicates that the C-terminal cleavage of 2A occurs at Q1398. 2B and 2C have sequence homology to kobuviral sequences, particularly in a conserved 2C helicase domain G1724SPGVGKS 198 that aligns to PF00910 RNA helicase domain.

Whereas no sequence homology to any picornavirus record in GenBank was found for TVHV 3A, 3B displays a conserved tyrosine in position 3 as well as a conserved glycine in position 5. The TVHV 3C protease contains the active site motif G2298MCGA consistent with 3C proteases of other picornaviruses and shows highest homology to cosaviral 3C(PF00548 3C cysteine protease (picornain) aligning to aa 2153-2321, FIG. 1). The identity of a 472 aa sequence at the 5′ end of the genome as TVHV 3D is supported by homology of aa 2355-2809 to PF00680 RNA-dependent RNA polymerases (RdRp), and the conservation of positive-strand viral RdRp motifs A-E (Poch et al., EMBO. 1989; 8:3867-74) (FIG. 1).

The assembled TVHV genome sequences were used to re-analyze the initial read library generated by unbiased high throughput pyrosequencing. This analysis confirmed the presence, and overlap with adjacent sequences, of divergent 2A and 3A region reads in the initial data set.

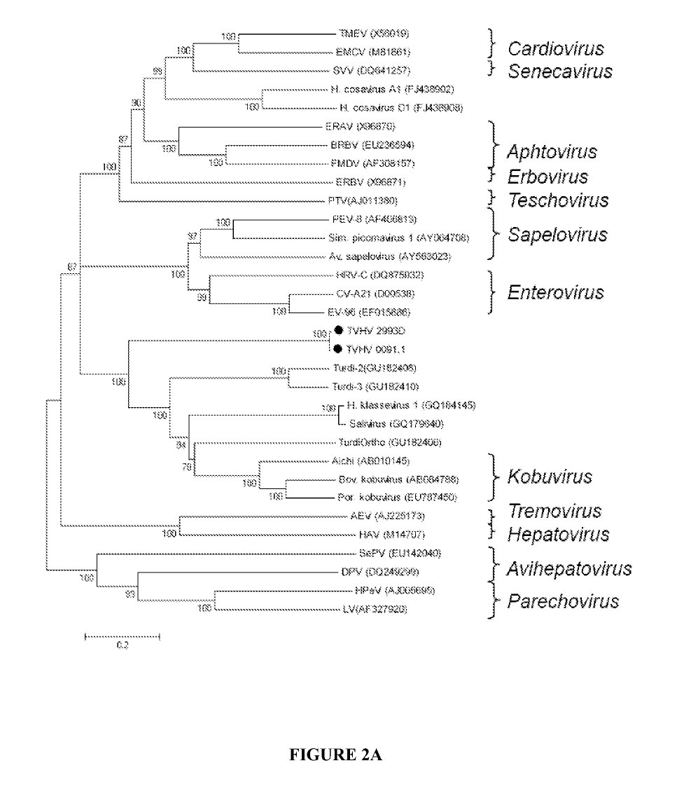

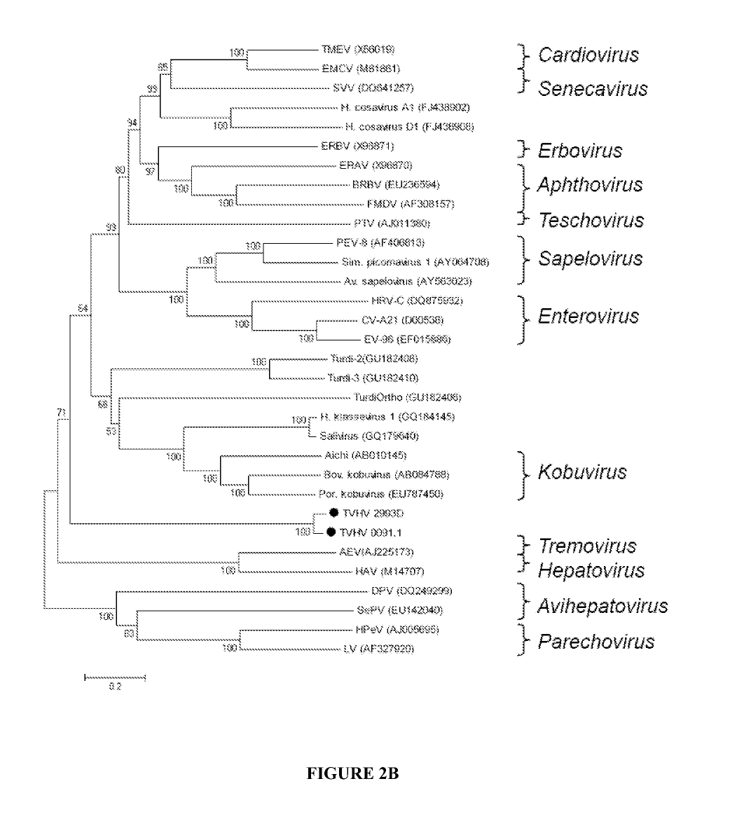

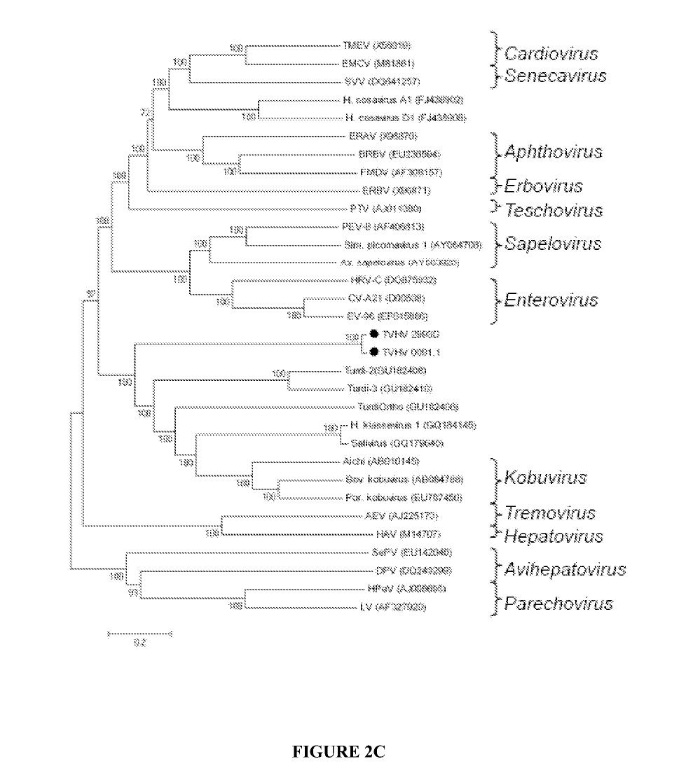

Phylogenetic analyses based on aa sequence of the most informative genome regions 2C/3C/3D, the P1 region, and the full polyprotein sequence excluding the non-conserved aa 799-1199, showed TVHV as a distinct species separate from classified genera (FIG. 2). The 2C/3C/3D analysis indicates TVHV in an ancestral position to kobu-, klasse-, and turdiviruses, the viruses most related to TVHV.

### Example 10

### TVHV RNA Load in Liver, Bile, Sera and Cloacal Swabs

Two TaqMan real-time PCR assays, both targeting the 5′UTR, were developed to quantitate viral RNA load in affected animals. Assay 1 (Table 1) was replaced by assay 2 (Table 2) following the characterization of additional 221 TVHV strains with divergent sequence.

In the liver samples from turkeys with TVH, viral RNA typically exceeded 105 copies/300 ng of total RNA, with only one animal showing a lower load (animal 0690, Table 1). No viral RNA was detected in livers from non-TVH control animals. Analysis of animals 1813.3 and 1999 indicated the presence of the virus in the intestine, and pancreas as well as the liver. Cloacal swabs, bile and sera from 28-, 29- and 39-day-old turkey poults with disease were also analyzed. Five of 9 cloacal swabs from TVH229 affected animals were positive for viral RNA (Table 2). Viral RNA exceeding 104 copies/300 ng total RNA was detected in the bile of 5 of 5 TVH cases tested. Viral RNA was also present in the sera of 8 of 18 TVH cases.

Cloacal swabs were analyzed from 32- and 39-day-old turkey poults that did not have TVH according to histopathology. In 2 of 10 animals viral RNA was detected (Table 2).

### Example 11

### Localization of TVHV RNA and Protein in Liver

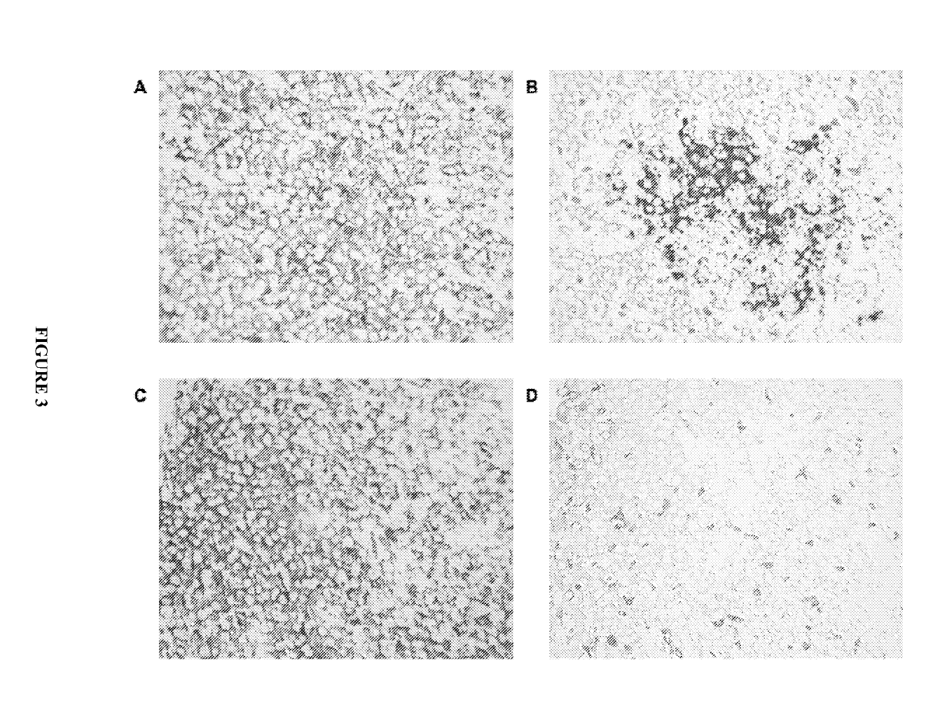

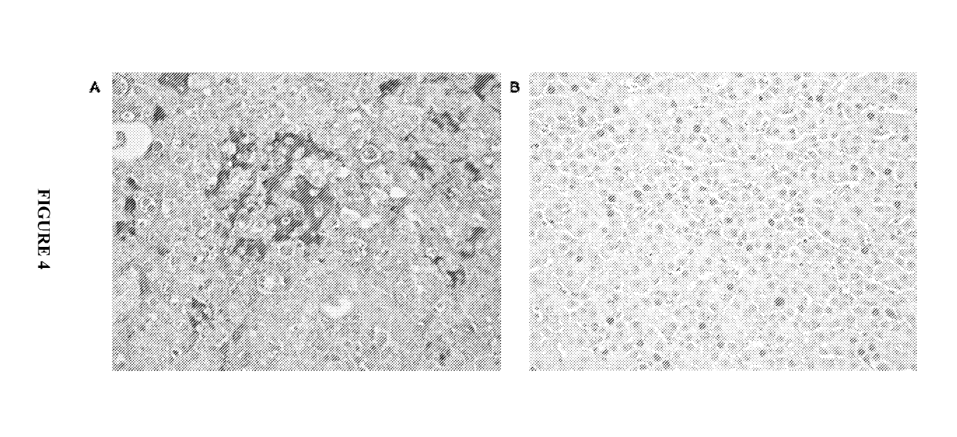

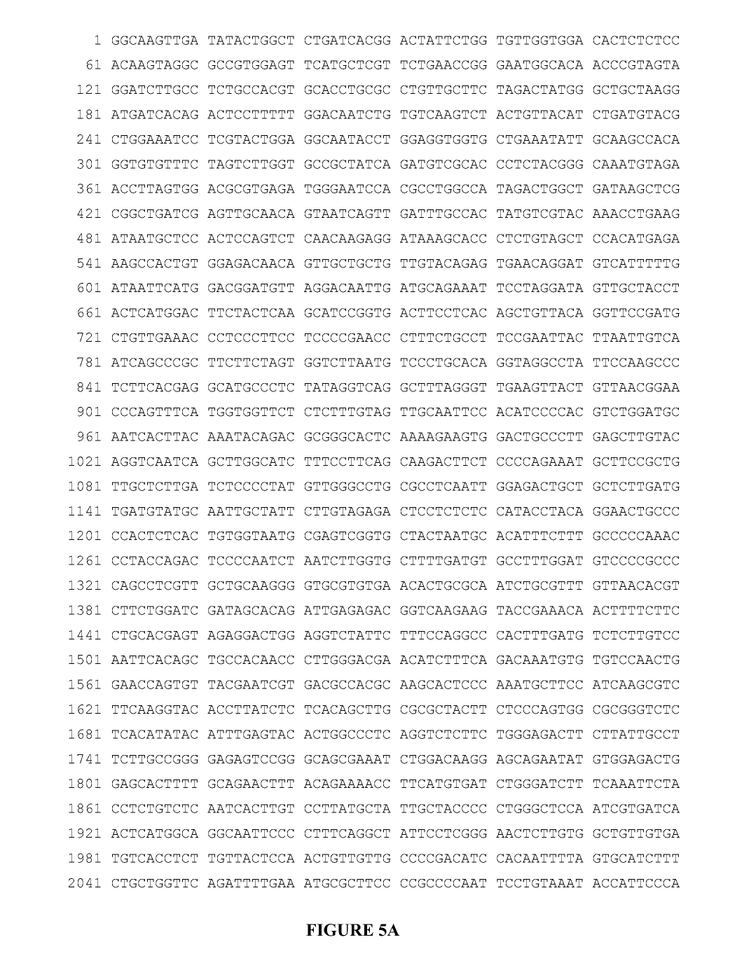

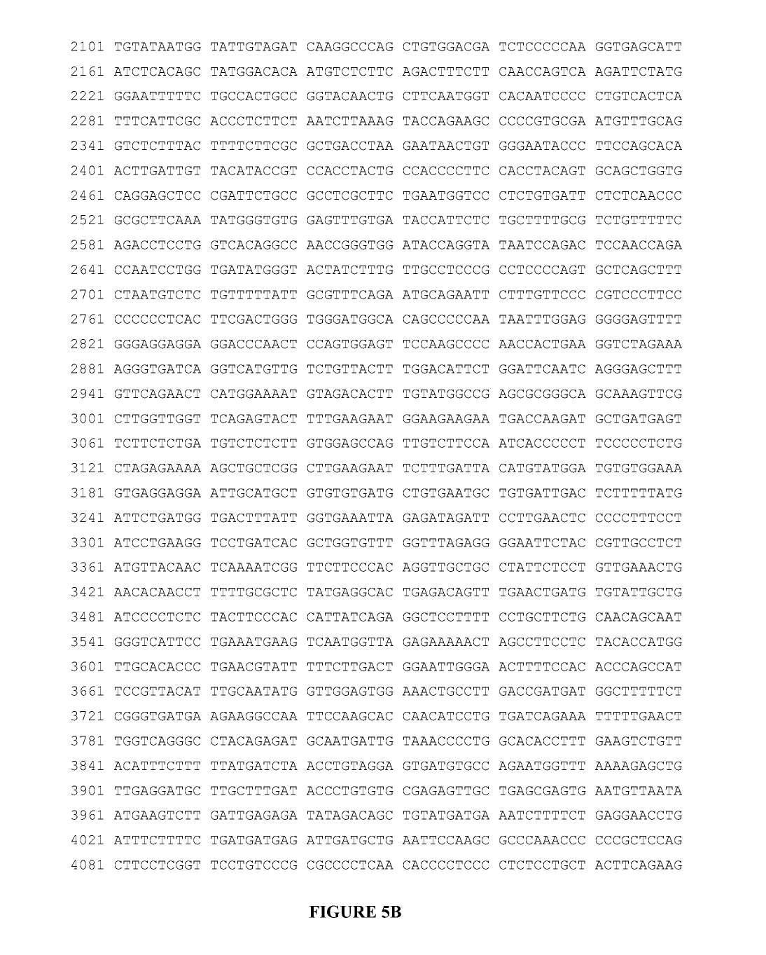

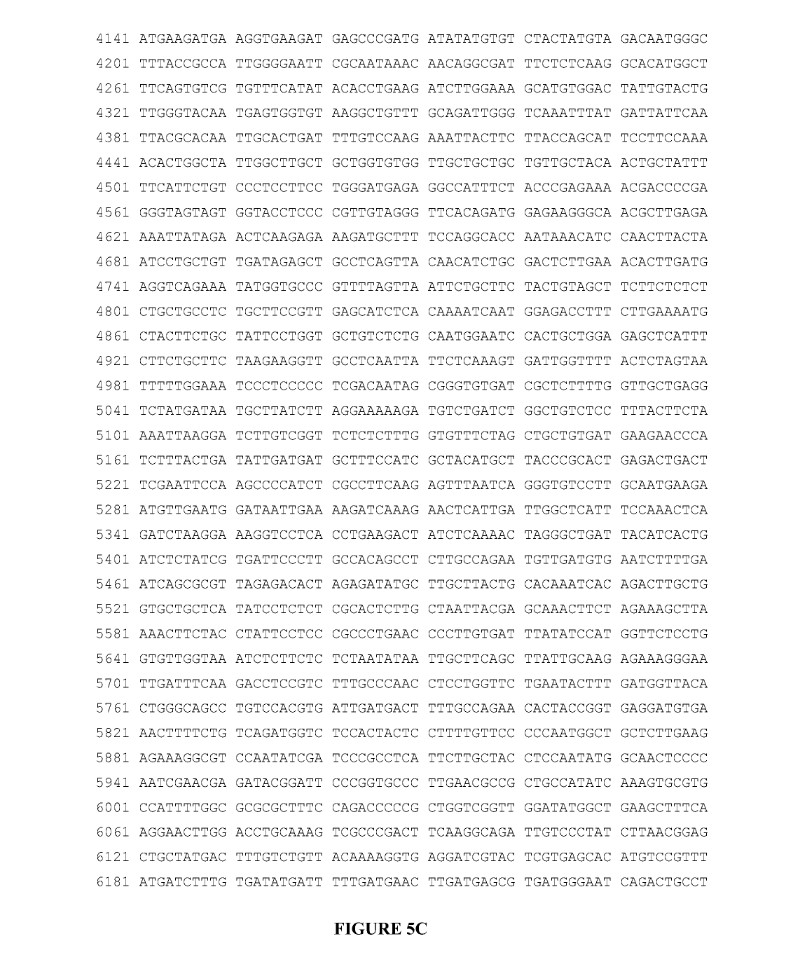

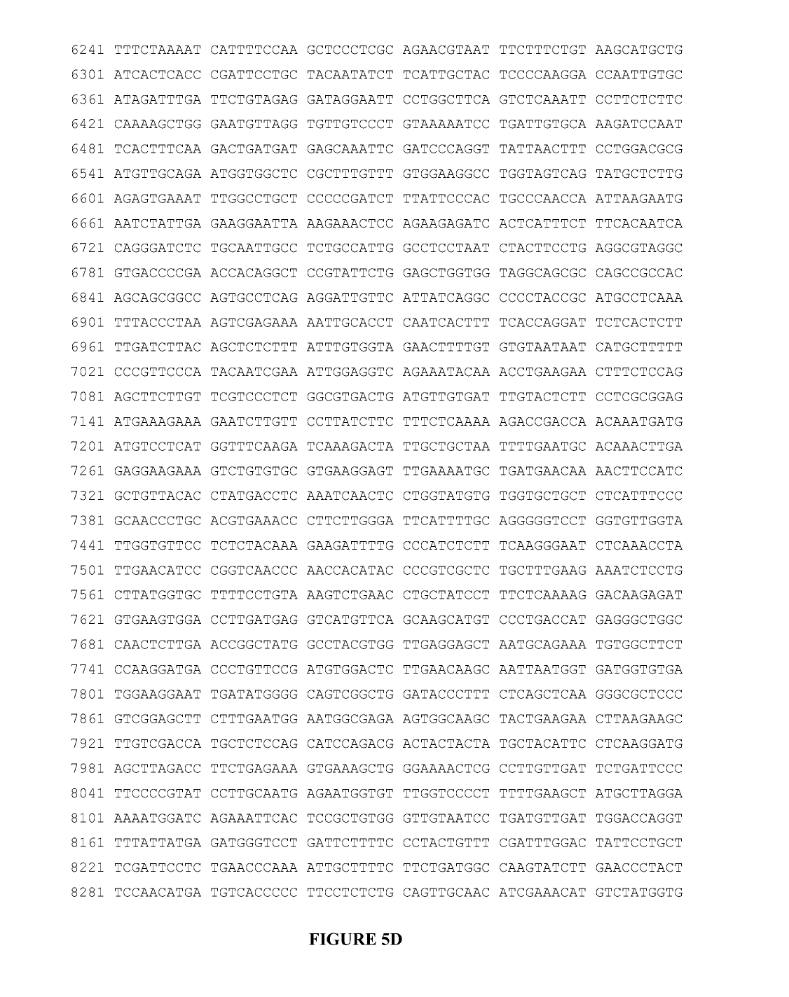

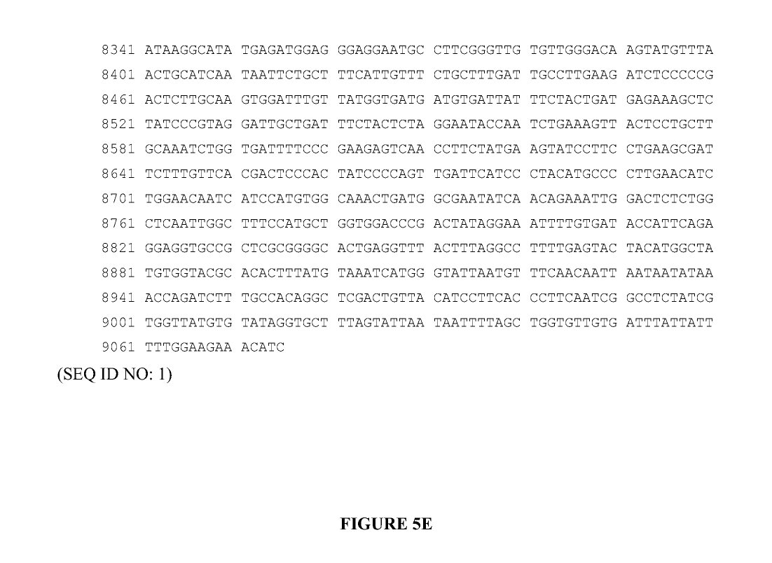

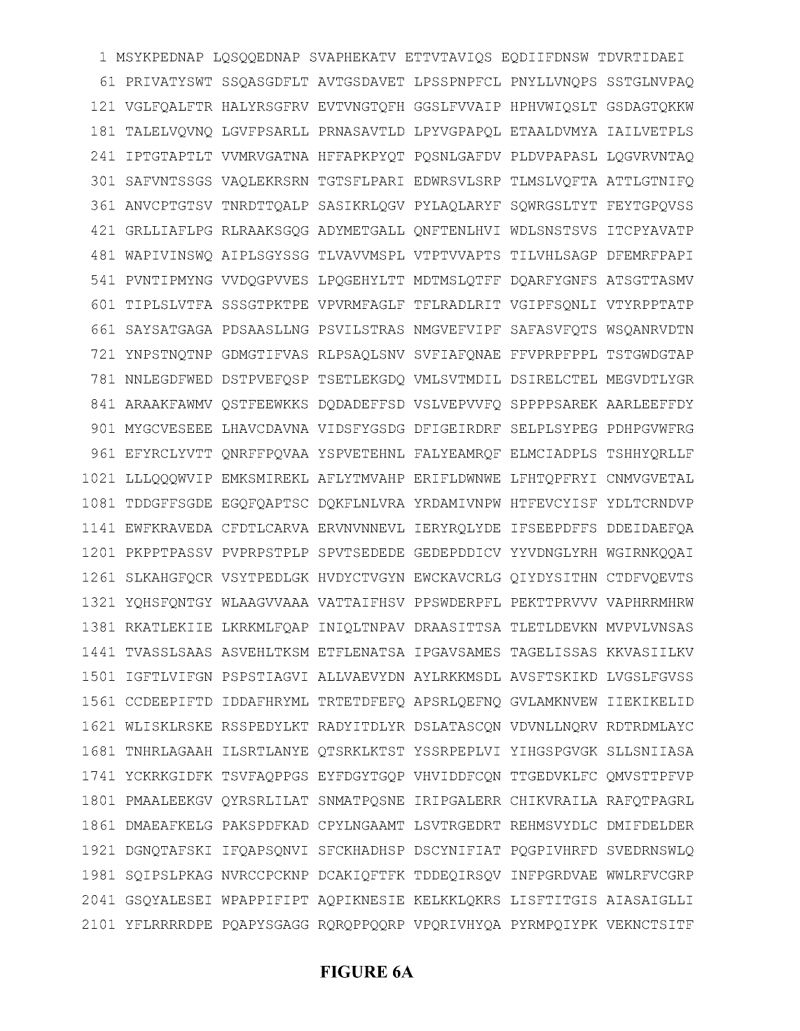

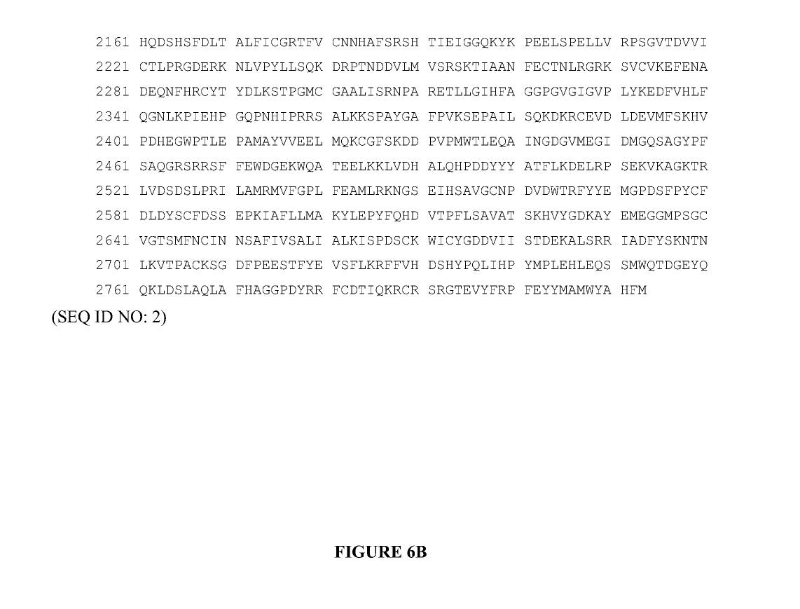

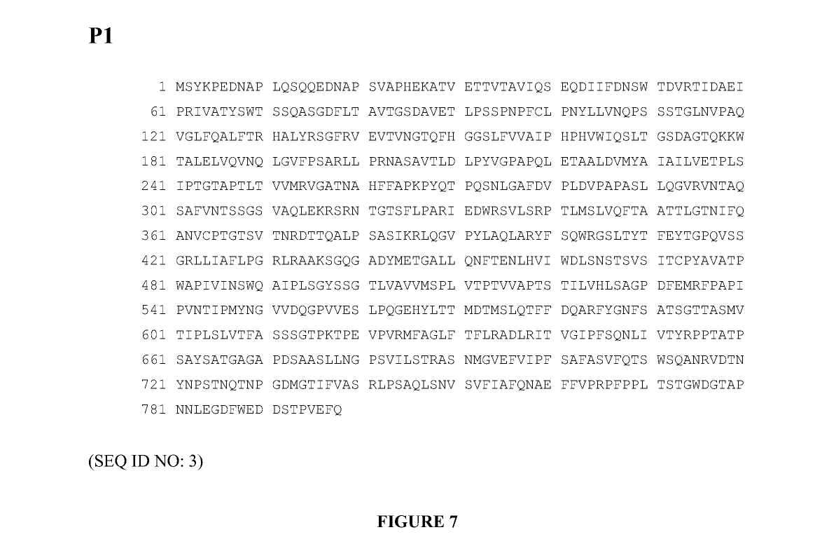

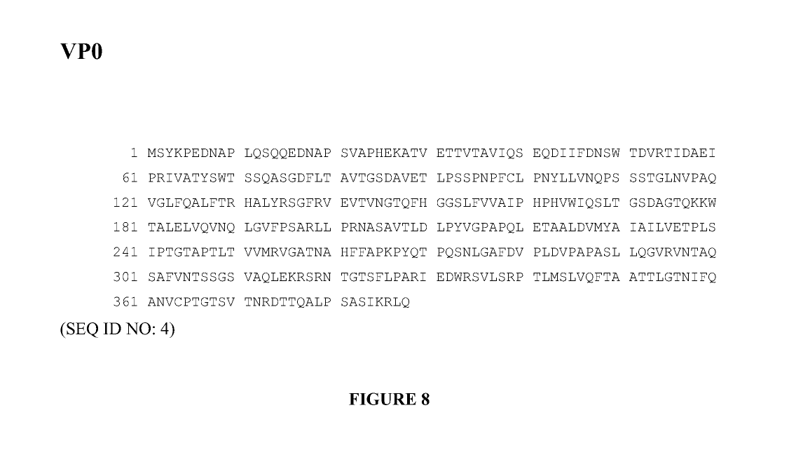

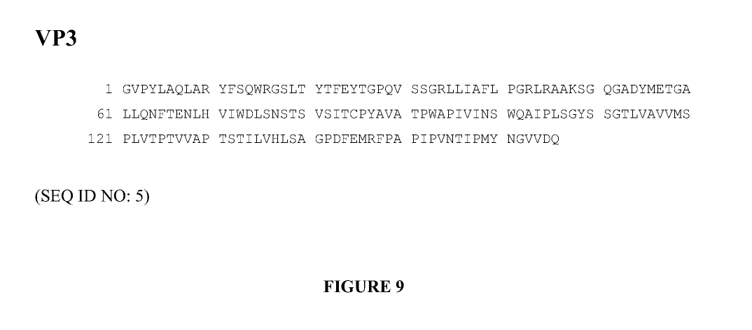

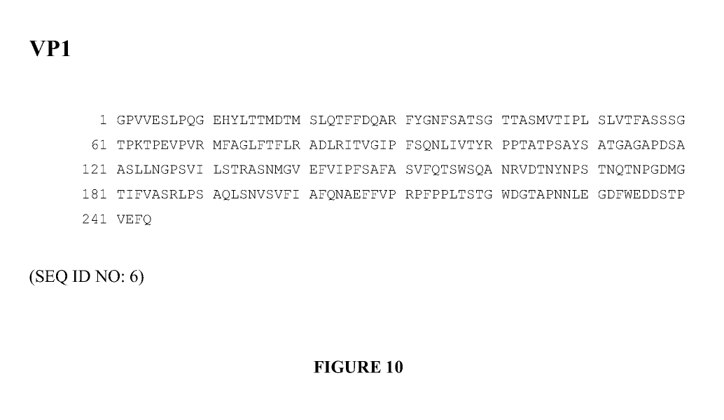

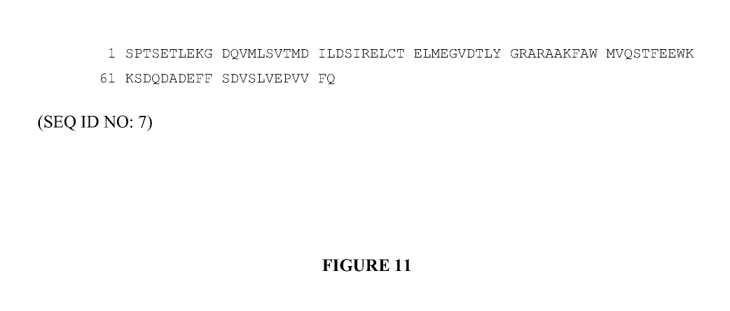

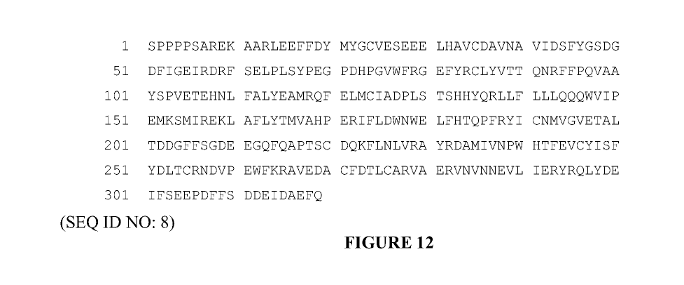

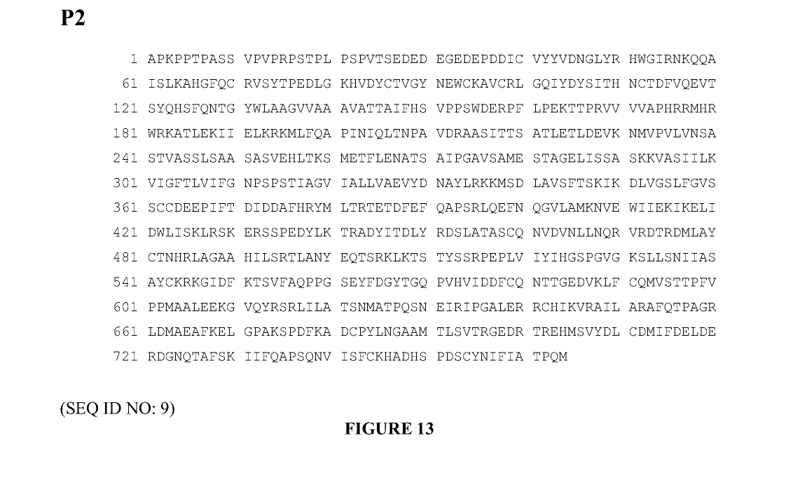

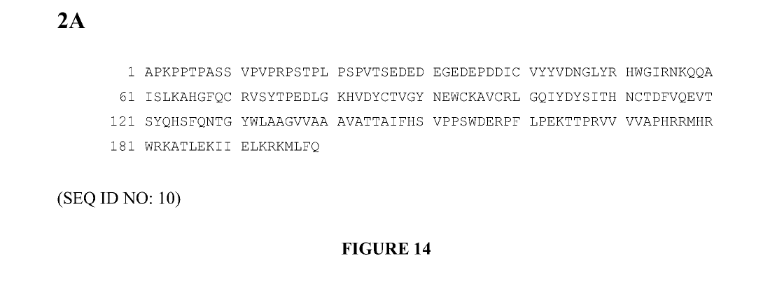

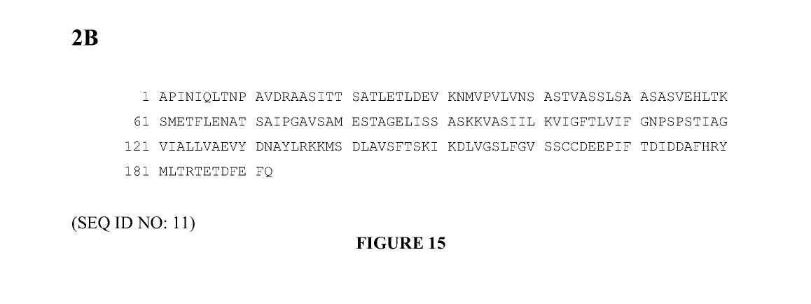

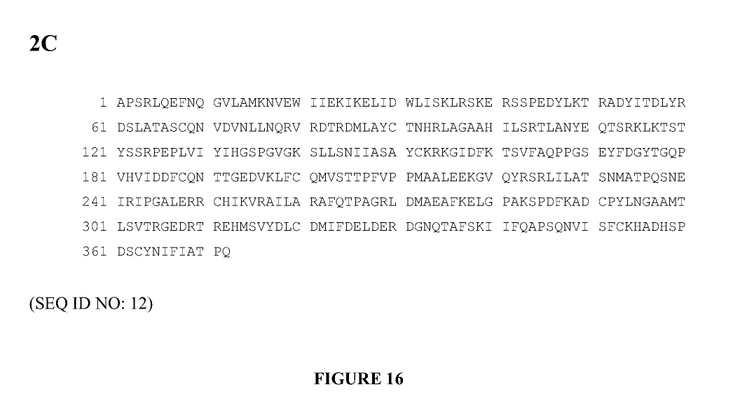

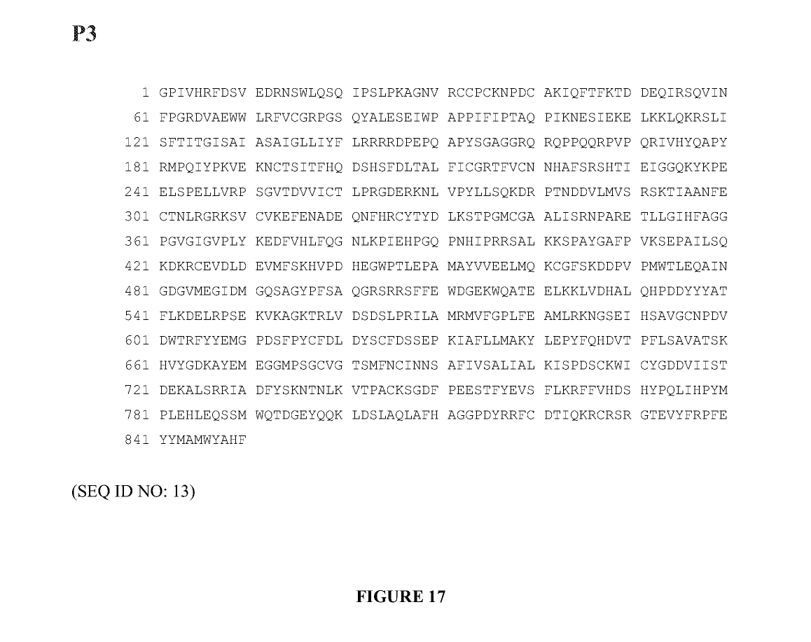

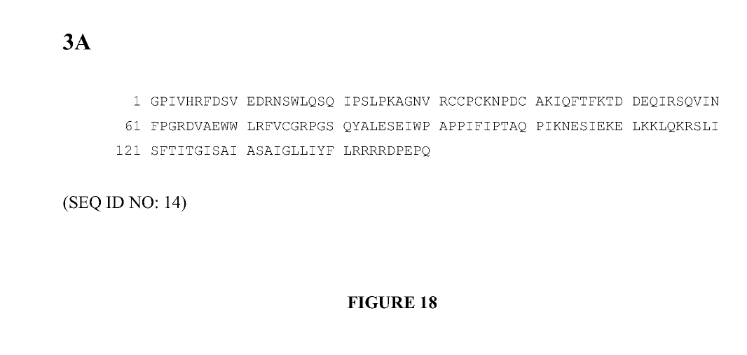

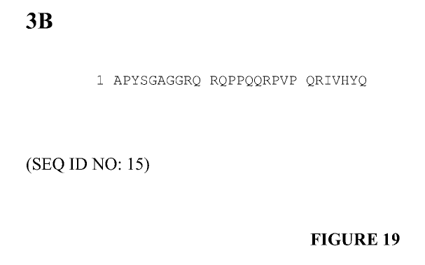

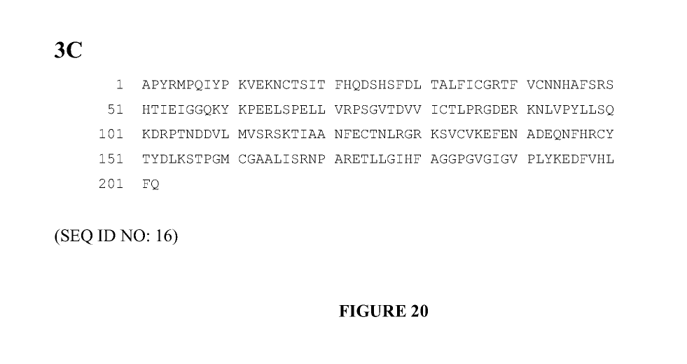

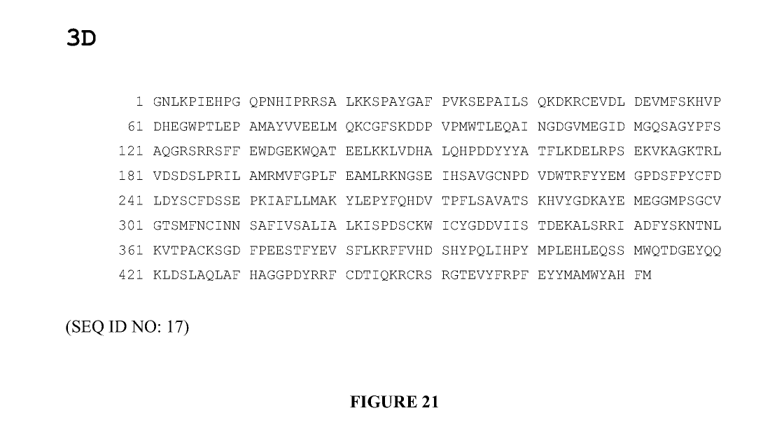

The distribution of TVHV RNA was examined in liver sections of affected and unaffected poults by in situ hybridization (ISH). TVHV signal was present in the cytoplasm of hepatocytes of affected poults. No signal was observed in normal poults (FIG. 3). Hybridization signal with a control β-actin probe was present in both affected and normal poults; however, β-actin signal was less pronounced in affected poults. ISH also showed the presence of viral RNA in an intestinal sample from one of the 0091 animals (not shown), in line with the real-time 243 PCR data obtained for animals 1813.3 and 1999 (see Table 1). Serum from TVH-affected poult 394.9 that was PCR-positive for TVHV RNA (see Table 2) was used on paraffin-embedded tissues for immunohistochemical analysis. The serum showed reactivity in livers from affected poults. No reactivity was observed with normal poults (FIG. 4)

Discussion

Phylogenetic analyses indicates that TVHV, although distantly related to viruses of the Kobu- and Klassevirus genera as well as to recently identified unclassified turdiviruses, is distinct from known picornaviruses.

TVHV contains a unique 2A region. TVHV encodes multiple 2A products. Although multiple 2A products have also been described for Ljungan virus, seal picornavirus, and duck hepatitis virus (Kapoor et al., J. Virol. 2008; 82:311-20; Johansson et al., J Gen Virol. 2003; 84:837-44; Tseng et al., Virus Res. 2007; 126:19-31), they are shorter than those predicted for TVHV (2A1: 20 aa in duck hepatitis virus vs 82 aa in TVHV, 2A2: 161 vs 320 aa, and 2A3: 124 vs 199 aa). Also remarkable is the lack of sequence homology of TVHV 3A to picorna- or other viral sequences. Whereas VP3 (representing P1) is closest to turdivirus 1 (30% aa identity), 2B/C (representing P2) is closer to Aichi virus (28%), and 3C/D (representing P3) is closer to turdivirus 3 (39%). Thus, TVHV shows classic features of known picornaviruses, 266 but also unique features that do not support inclusion of TVHV into existing taxa of the Picornaviridae family.

Turkey poults with TVH may have diarrhea and pancreatitis as well as hepatitis. Picornaviral particles have been described in the feces of animals with TVH (Kapoor et al., Proc Natl Acad Sci USA. 2008; 105:20482-7; Holtz et al., Virol J. 2008; 5:159). Accordingly, TVHV RNA was detected in the intestine, pancreas, bile and cloacal swabs as well as the liver. These findings are consistent with a fecal-oral route for transmission. TVHV RNA was also detected in the cloacal swabs of 2 of 10 asymptomatic poults. As these animals were housed on a farm with history of TVH, this finding is consistent with reports suggesting subclinical infections (Klein et al, Avian Dis. 1991; 35:115-25; Andral and Toquin. Avian Pathol. 1984; 13:377-88; Andral et al., Avian Pathol. 1990; 19:245-54). The advent of a non invasive screening test for TVHV may aid in disease containment.

The results described herein molecularly characterizes a picornavirus in turkey viral hepatitis and links it to disease through measurements of load and tissue distribution, viremia and a humoral immune response to the agent. The results described herein show that TVHV represents a new species in the order Picornavirales and an important candidate for causing TVH.

### REFERENCES

1. Klein P N, Castro A E, Meteyer C U, Reynolds B, Swartzman-Andert J A, Cooper G, et al. Experimental transmission of turkey viral hepatitis to day-old poults and identification of associated viral particles resembling picornaviruses. Avian Dis. 1991; 35:115-25.
2. Mongeau J D, Truscott R B, Ferguson A E, Connell M C. Virus hepatitis in turkeys. Avian Dis. 1959; 3:388-96.
3. Snoeyenbos G H, Basch H I, Sevoian M. An infectious agent producing hepatitis in turkeys. Avian Dis. 1959; 3:377-88.
4. Snoeyenbos G H, Basch H I. Further studies of virus hepatitis of turkeys. Avian Dis. 1960; 3:477-84.
5. Tzianabos T, Snoeyenbos G H. Some physiochemical properties of turkey hepatitis virus. Avian Dis. 1965; 9:152-6.
6. MacDonald J W, Randall C J, Dagless M D, Gray E W. Picorna-like virus causing hepatitis and pancreatitis in turkeys. Vet. Rec. 1982; 111:323.
7. Cho B R. An adenovirus from a turkey pathogenic to both chicks and turkey poults. Avian Dis. 1976; 20:714-23.
8. Wages D P, Ficken M D. Cryptosporidiosis and turkey viral hepatitis in turkeys. Avian Dis. 1989; 33:191-4.
9. Palacios G, Quan P L, Jabado O J, Conlan S, Hirschberg D L, Liu Y, et al. Panmicrobial oligonucleotide array for diagnosis of infectious diseases. Emerg Infect Dis. 2007; 13:73-81.
10. Tamura K, Dudley J, Nei M, Kumar S. MEGA4: Molecular Evolutionary Genetics Analysis (MEGA) software version 4.0. Molecular Biology and Evolution. 2007; 24:1596-9.
11. Wutz G, Auer H, Nowotny N, Grosse B, Skern T, Kuechler E. Equine rhinovirus serotypes 1 and 2: relationship to each other and to aphthoviruses and cardioviruses. J. Gen Virol. 1996; 77:1719-30.
12. Woo P C, Lau S K, Huang Y, Lam C S, Poon R W, Tsoi H W, et al. Comparative analysis of six genome sequences of three novel picornaviruses, turdiviruses 1, 2 and 3, in dead wild birds and proposal of two novel genera, Orthoturdivirus and Paraturdivirus, in Picornaviridae. J Gen Virol. 2010: Jun. 16. [Epub ahead of print]
13. Kapoor A, Victoria J, Simmonds P, Wang C, Shafer R W, Nims R et al. A highly divergent picornavirus in a marine mammal. J. Virol. 2008; 82:311-20.
14. Johansson E S, Niklasson B, Tesh R B, Shafren D R, Travassos da Rosa A P, Lindberg A M. Molecular characterization of M1146, an American isolate of Ljungan virus (LV) reveals the presence of a new LV genotype. J Gen Virol. 2003; 84:837-44.
15. Tseng C H, Tsai H J. Molecular characterization of a new serotype of duck hepatitis virus. Virus Res. 2007; 126:19-31.
16. Poch O, Sauvaget I, Delarue M, Tordo N. Identification of four conserved motifs among the RNA-dependent polymerase encoding elements. EMBO. 1989; 8:3867-74.
17. Greninger A L, Runckel C, Chiu C Y, Haggerty T, Parsonnet, J, Ganem D, et al. The complete genome of klassevirus—a novel picornavirus in pediatric stool. Virol J. 2009; 6:82.
18. Holtz L R, Finkbeiner S R, Zhao G, Kirkwood C D, Girones R, Pipas J M, et al. Klassevirus 1, a previously undescribed member of the family Picornaviridae, is globally widespread. Virol J. 2009; 6:86.
19. Li L, Victoria J, Kapoor A, Blinkova O, Wang C, Babrzadeh F, et al. A novel picornavirus associated with gastroenteritis. J. Virol. 2009; 83:12002-6.
20. Kapoor A, Victoria J, Simmonds P, Slikas 363 E, Chieochansin T, Naeem A, et al. A highly prevalent and genetically diversified Picornaviridae genus in South Asian children. Proc Natl Acad Sci USA. 2008; 105:20482-7.
21. Holtz L R, Finkbeiner S R, Kirkwood C D, Wang D. Identification of a novel picornavirus related to cosaviruses in a child with acute diarrhea. Virol J. 2008; 5:159.
22. Andral B, Toquin D. Observations and isolation of pseudopicornavirus from sick turkeys. Avian Pathol. 1984; 13:377-88.
23. Andral B, Lagadic M, Bennejean G, Toquin D, Florent J M. Picorna-like viruses of young turkeys: pathogenesis of a disease of poults caused by a picorna-like virus. Avian Pathol. 1990; 19:245-54.

What is claimed is:

1. An isolated nucleic acid having a sequence of SEQ ID NO: 1.

2. An isolated nucleic acid which comprises 10 consecutive nucleotides having a sequence of SEQ ID NO: 1.

3. An isolated nucleic acid which is a variant of any one of SEQ ID NO: 1 and has at least about 85% identity to SEQ ID NO: 1.

4. The isolated nucleic acid of claim 3, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1.

5. An isolated nucleic acid complementary to a sequence of SEQ ID NO: 1.

6. An isolated nucleic acid which comprises 10 consecutive nucleotides complementary to a sequence of SEQ ID NO: 1

7. An isolated nucleic acid which is a complementary to a variant of any one of SEQ ID NO: 1 and wherein the variant has at least about 85% identity to SEQ ID NO: 1.

8. The isolated nucleic acid of claim 5, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1.

9. The isolated nucleic acid of claim 2 or claim 5, wherein the identity is determined by analysis with a sequence comparison algorithm.

10. The isolated nucleic acid of claim 8, wherein the sequence comparison algorithm is FASTA version 3.0t78 using default parameters.

11. An isolated polypeptide having a sequence selected from the group consisting of: SEQ ID NOs: 2-17.

12. An isolated polypeptide which comprises 8 consecutive amino acids having a sequence selected from the group consisting of: SEQ ID NOs: 2-17.

13. An isolated polypeptide which is a variant of any one of SEQ ID NOs: 2-17 and has at least about 85% identity to SEQ ID NO: 2-17.

14. The isolated polypeptide acid of claim 13, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 2-17.

15. The isolated polypeptide of claim 14, wherein the identity is determined by analysis with a sequence comparison algorithm.

16. The isolated polypeptide of claim 14, wherein the sequence comparison algorithm is FASTA version 3.0t78 using default parameters.

17. An oligonucleotide probe which comprises from about 10 nucleotides to about 50 nucleotides, wherein at least about 10 contiguous nucleotides are at least 95% complementary to a nucleic acid target region within a nucleic acid sequence of SEQ ID NO: 1.

18. The oligonucleotide of claim 17, wherein the probe is at least about 95.5%, about 96%, about 96.5%, about 97%, about 97.5%, about 98%, about 98.5%, about 99%, about 99.5% or about 99.9% complementary to SEQ ID NO: 1.

19. The oligonucleotide of claim 18, wherein the oligonucleotide probe consists essentially of from about 10 to about 50 nucleotides.

20. A synthetic nucleic acid which has a sequence consisting of from about 10 to about 30 consecutive nucleotides from a nucleic acid sequence of SEQ ID NO: 1.

21. A method for determining the presence or absence of TVHV in a biological sample, the method comprising:
a) contacting nucleic acid from a biological sample with at least one primer which is a nucleic acid of claim 17,
b) subjecting the nucleic acid and the primer to amplification conditions, and
c) determining the presence or absence of amplification product, wherein the presence of amplification product indicates the presence of RNA associated with TVHV in the sample.

22. A synthetic nucleic acid which has a sequence consisting of from about 10 to about 30 consecutive nucleotides from a nucleic acid sequence which is complementary to a nucleic acid sequence of SEQ ID NO: 1.

23. A method for determining the presence or absence of TVHV in a biological sample, the method comprising:
a) contacting nucleic acid from a biological sample with at least one primer which is a nucleic acid of claim 17, and
b) subjecting the nucleic acid and the primer to amplification conditions, and
c) determining the presence or absence of amplification product, wherein the presence of amplification product indicates the presence of RNA associated with TVHV in the sample.

24. A primer set for determining the presence or absence of TVHV in a biological sample, wherein the primer set comprises at least one synthetic nucleic acid sequence selected from the group consisting of:
a) a synthetic nucleic acid which has a sequence consisting of from about 10 to about 30 consecutive nucleotides from a nucleic acid sequence of SEQ ID NO: 1, and
b) a synthetic nucleic acid which has a sequence consisting of from about 10 to about 30 consecutive nucleotides from a nucleic acids sequence which is complementary to a nucleic acid sequence of SEQ ID NO: 1.

25. A method for determining whether or not a sample contains TVHV, the method comprising:
a) contacting a biological sample with an antibody that specifically binds a polypeptide encoded by the nucleic sequence acid of any one of SEQ ID NO: 1, and
b) determining whether or not the antibody binds to an antigen in the biological sample, wherein binding indicates that the biological sample contains TVHV.

26. The method of claim 25, wherein the determining comprises use of a lateral flow assay or ELISA.

27. A method for determining whether or not a biological sample has been infected by TVHV, the method comprising:
a) determining whether or not a biological sample contains antibody that specifically binds a polypeptide encoded by the nucleic sequence acid of any one of SEQ ID NO: 1.

28. An interfering RNA (iRNA) comprising a sense strand having at least 15 contiguous nucleotides complementary to the anti-sense strand of a gene from a virus comprising a nucleic acid sequence of SEQ ID NO: 1.

29. An interfering RNA (iRNA) comprising an anti-sense strand having at least 15 contiguous nucleotides complementary to the sense strand of gene from a virus comprising a nucleic acid sequence of SEQ ID NO: 1.

30. A method for reducing the levels of a viral protein, viral mRNA or viral titer in a cell in an animal comprising: administering an iRNA agent to an animal, wherein the iRNA agent comprises a sense strand having at least 15 contiguous nucleotides complementary to gene from a TVHV comprising a nucleic acid sequence of SEQ ID NO: 1 and an antisense strand having at least 15 contiguous nucleotides complementary to the sense strand.

31. The method of claim 30, further comprising co-administering a second iRNA agent to the animal, wherein the second iRNA agent comprises a sense strand having at least 15 or more contiguous nucleotides complementary to second gene from the TVHV comprising a nucleic acid sequence of SEQ ID NO: 1 and an antisense strand having at least 15 or more contiguous nucleotides complementary to the sense strand.

32. A method of reducing the levels of a viral protein from at least one gene of a TVHV in a cell in an animal, the method comprising administering an iRNA agent to an animal, wherein the iRNA agent comprises a sense strand having at least 15 or more contiguous from a nucleic acid sequence of SEQ ID NO: 1 complementary to a gene from a TVHV and an antisense strand having at least 15 or more contiguous nucleotides complementary to the sense strand of a nucleic acid sequence of SEQ ID NO: 1.

33. The method of any of claim 21, 23, 25, 25 or 27, wherein the sample is from a avian.

34. The method of claim 33, wherein the avian is a turkey.

35. An isolated virus comprising any one of the nucleic acid sequences of SEQ ID NO: 1.

36. An isolated virus comprising a polypeptide encoded by the nucleic sequence acid of any one of SEQ ID NO: 1.

37. A TVHV immunogenic composition comprising a TVHV nucleic acid.

38. The immunogenic composition of claim 37, wherein the TVHV nucleic acid is a nucleic acid sequence of SEQ ID NO: 1.

39. The immunogenic composition of claim 37, wherein the TVHV nucleic acid comprises least 24 consecutive nucleic acids of SEQ ID NO: 1.

40. The immunogenic composition of claim 37, wherein the TVHV nucleic acid is substantially identical to the nucleic acid sequence of SEQ ID NO: 1.

41. The immunogenic composition of claim 37, wherein the TVHV nucleic acid is a variant of SEQ ID NO: 1 having at least about 85% identity to SEQ ID NO: 1.

42. The nucleic acid of claim 41, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1.

43. A TVHV immunogenic composition comprising a TVHV polypeptide.

44. The immunogenic composition of claim 43, wherein the TVHV polypeptide is a polypeptide encoded by any of SEQ ID NO: 1.

45. The immunogenic composition of claim 43, wherein the TVHV polypeptide is a polypeptide encoded by a nucleic acid comprising least 24 consecutive nucleic acids of SEQ ID NO: 1.

46. The immunogenic composition of claim 43, wherein the TVHV polypeptide is a polypeptide encoded by a nucleic acid that is substantially identical to the nucleic acid sequence of SEQ ID NO: 1.

47. The immunogenic composition of claim 43, wherein the TVHV polypeptide is a polypeptide encoded by a nucleic acid that is a variant of SEQ ID NO: 1 having at least about 85% identity to SEQ ID NO: 1.

48. The polypeptide of claim 47, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1.

49. The immunogenic composition of claim 43, wherein the TVHV polypeptide is a polypeptide comprising the amino acid sequence of SEQ ID NOs: 2-17.

50. The immunogenic composition of claim 43, wherein the TVHV polypeptide is a polypeptide comprising least 8 consecutive amino acids of any of SEQ ID NOs: 2-17.

51. The immunogenic composition of claim 43, wherein the TVHV polypeptide is substantially identical to the amino acid sequence of any of SEQ ID NOs: 2-17.

52. The immunogenic composition of claim 43, wherein the TVHV polypeptide is a variant of any of SEQ ID NOs: 2-17 and having at least about 85% identity to SEQ ID NOs: 2-17.

53. The polypeptide of claim 52, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NOs: 2-17.

54. An isolated antibody that specifically binds to a polypeptide encoded by the nucleotide sequence shown in any one of SEQ ID NO: 1.

55. An isolated antibody that specifically binds to a polypeptide having the sequence of any of SEQ ID NO: 2-17.

56. The antibody of claim 54 or 55, wherein the antibody binds a TVHV or a TVHV polypeptide and inhibits, neutralizes or reduces the function or activity of the TVHV or TVHV polypeptide.

57. The antibody of claim 54 or 55, wherein the antibody is a polyclonal antibody.

58. The antibody of claim 54 or 55, wherein the antibody is a monoclonal antibody.

59. The antibody of claim 54 or 55, wherein the antibody is an avian antibody.

60. The antibody of claim 54 or 55, wherein the antibody is a turkey antibody.

61. The antibody of claim 54 or 55, wherein the antibody is an IgM antibody.

62. The antibody of claim 54 or 55, wherein the antibody is a chimeric antibody.

63. An immunogenic composition comprising a killed virus comprising a TVHV polypeptide.

64. An immunogenic composition comprising an attenuated virus comprising a TVHV polypeptide.

65. The immunogenic composition of any of claim 37, 43, 63 or 64 further comprising at least one excipient, additive or adjuvant.

66. A method of inducing an immune response in an animal, the method comprising administering the TVHV immunogenic composition of any of claim 37, 43, 63 or 64.

67. A method for preventing, or reducing TVHV infection in an animal, the method comprising administering to the animal the TVHV immunogenic composition of any of claim 37, 43, 63 or 64.

68. A method for preventing, or reducing TVHV infection in an animal, the method comprising administering to the animal the antibody of claim 54 or 55.

69. The method of any of claim 66, 67 or 68, wherein the administration is oral administration or injection administration.

70. Use of the immunogenic composition of any of claim 37, 43, 63, or 64 in the manufacture of a vaccine for the treatment of condition TVHV infection in an animal.
